(19) United States
(12) Patent Application Publication
(10) Pub. No.: US 2008/0267989 A1
(43) Pub. Date: Oct. 30, 2008

(54) Hiv Gp-41-Membrane Proximal Region Arrayed On Hepatitis B Surface Antigen Particles as Novel Antigens

(75) Inventors: Sanjay K. Phogat, Frederick, MD (US); Richard Wyatt, Rockville, MD (US); Ira Berkower, Washington, DC (US)

Correspondence Address: KLARQUIST SPARKMAN, LLP, 121 S.W. SALMON STREET, SUITE #1600, PORTLAND, OR, 97204-2988, US

(73) Assignee: THE GOVERNMENT OF THE UNITED STATES OF AMERICA as, Rockville, MD (US)

(21) Appl. No.: 11/816,069

(22) PCT Filed: Feb. 17, 2006
(86) PCT No.: PCT/US06/05613

§ 371 Date: Aug. 10, 2007

Related U.S. Application Data
(60) Provisional application No. 60/653,930, filed on Feb. 18, 2005.

(51) Int. Cl. A61K 39/00; C07K 14/00; C12N 15/11; C12N 5/06; C12Q 1/70; A61P 37/00; C12Q 1/02; C12N 7/00; A61K 35/76; A61K 38/00

(52) U.S. Cl. 424/188.1; 530/324; 530/350; 536/23.4; 435/325; 435/235.1; 424/93.6; 514/12; 435/5; 435/29

(57) ABSTRACT

Recombinant HBsAg-gp120 has been used to present approximately amino acids 1-500 gp120. However, this presentation of gp120 in this form has not successfully been used to produce neutralizing antibodies. The use of the immunogenic Hepatitis B surface antigen (HBsAg) particulate platform to array specific epitopes from the conserved, neutralization-sensitive membrane proximal region (MPR) of HIV-1, and the use of these monomeric fusion proteins, polymeric forms of these fusion proteins, and nucleic acids encoding these fusion proteins to induce an immune response to HIV-1 are disclosed.

### CROSS REFERENCE TO RELATED APPLICATION

This application claims the benefit of U.S. Provisional Application No. 60/653,930, filed on Feb. 18, 2005, which is incorporated herein by reference.

### FIELD

This application relates to the field of human immunodeficiency virus, specifically to the use of epitopes of glycoprotein 41 (gp41) to induce an immune response, including a protective immune response.

### BACKGROUND

Acquired immune deficiency syndrome (AIDS) is recognized as one of the greatest health threats facing modern medicine. Treatment of HIV-infected individuals as well as the development of vaccines to protect against infection are urgently needed. One difficulty has been in eliciting neutralizing antibodies to the virus.

The HIV-1 envelope glycoproteins (gp120-gp41), which mediate receptor binding and entry, are the major targets for neutralizing antibodies. Although the envelope glycoproteins are immunogenic and induce a variety of antibodies, the neutralizing antibodies that are induced are strain-specific, and the majority of the immune response is diverted to non-neutralizing determinants (Weiss, R. A., et al., Nature, 1985. 316 (6023): p. 69-72; Wyatt, R. and J. Sodroski, Science, 1998. 280 (5371): p. 1884-8). Broadly neutralizing antibodies have been isolated only rarely from natural HIV infection and rarely, as only five broad-neutralizing antibodies have been identified to date. Three are gp41-directed (2F5, 4E10 and Z13) and the other two (b12 and 2G12) are gp120-directed. The three gp41 neutralizing antibodies recognize the membrane proximal region (MPR) of the HIV-1 gp41 glycoprotein. The MPR is roughly the 30 amino acids immediately upstream of the transmembrane region, is highly hydrophobic (50% of residues are hydrophobic), and is highly conserved across many HIV clades (Zwick, M. B., et al., J Virol, 2001. 75 (22): p. 10892-905). Recently the hydrophobic context of MPR and the presence of lipid membrane were shown to be important for the optimal binding of 2F5 and 4E10 antibodies (Ofek, G., et al., J Virol, 2004. 78 (19): p. 10724-37).

To date, immunization with conserved membrane proximal elements or the core 2F5 epitope in a number of contexts has failed to elicit broadly neutralizing antibodies (Coeffier, E., et al., Vaccine, 2000. 19 (7-8): 684-93; Eckhart, L., et al., J Gen Virol, 1996. 77 (Pt 9): 2001-8; Ernst, W., et al., Nucleic Acids Res, 1998. 26 (7): 1718-23; Ho, J., et al., Vaccine, 2002. 20 (7-8): 1169-80; Liang, X., et al., Epitop Vaccine, 1999. 17 (22): 2862-72; Liao, M., et al., Peptides, 2000. 21 (4): 463-8; Xiao, Y., et al., Immunol Invest, 2000. 29 (1): 41-50). Thus, there remains a need to identify HIV antigens that can be used to induce a protective immune response.

### SUMMARY

Historically, compositions used to produce an immune response against viral antigens include live-attenuated or chemically inactivated forms of the virus. However, this approach has limited utility when used for human immunodeficiency virus. Disclosed herein is the use of the immunogenic Hepatitis B surface antigen (HBsAg) platform to array epitopes from the conserved, neutralization-sensitive membrane proximal region (MPR) of HIV-1, and the use of this platform to induce an immune response to HIV-1.

In one embodiment, monomeric fusion proteins are disclosed. These proteins may include the following elements linked in an N-terminal to C-terminal direction: (a) a hepatitis B surface antigen; (b) a linear linking peptide; and, (c) an antigenic polypeptide comprising the amino acid sequence of SEQ ID NO:1, wherein the antigenic peptide is between 28 and 150 amino acids in length, wherein X1, X2 and X3 are any amino acid, and wherein a plurality of the monomeric fusion proteins form a self-aggregating multimeric ring structure upon expression in a host cell. Specific non-limiting examples of host cells include mammalian, insect, and yeast cells.

In additional embodiments, these proteins can include the following elements linked in an N-terminal to C-terminal direction: (a) a hepatitis B surface antigen; (b) a linear linking peptide; and, (c) an antigenic polypeptide comprising one to five repeats of the amino acid sequence of SEQ ID NO:24, wherein the antigenic polypeptide does not include amino acids 1 to 500 of a gp160 amino acid sequence (SEQ ID NO:25), and wherein X is any amino acid. The monomeric fusion proteins may further include basic or hydrophobic amino acid residues at the C-terminus and/or one or more HIV-specific T-helper cell epitopes. Viral-like particles including the fusion proteins are also provided herein.

Isolated nucleic acid molecules encoding the monomeric fusion proteins are also provided, as well as host cells transformed with the nucleic acid molecules and viral-like particles produced by the transformed host cells. Compositions comprising the viral-like particles are also provided.

The monomeric fusion proteins and polymeric forms thereof can be used to induce an immune response, such as a protective immune response, when introduced into a subject. The monomeric fusion proteins and polymeric forms thereof can also be used in assays to diagnose an HIV infection. Thus, methods are provided for inhibiting HIV infection in a subject, for inducing an immune response to HIV in a subject, for diagnosing HIV infection in a subject, and for identifying a B cell that produces antibodies that bind to gp41.

The foregoing and other objects, features, and advantages of the invention will become more apparent from the following detailed description, which proceeds with reference to the accompanying figures.

### BRIEF DESCRIPTION OF SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. All sequence database accession numbers referenced herein are understood to refer to the version of the sequence identified by that accession number as it was available on the designated date. In the accompanying sequence listing:

SEQ ID NO:1 is a consensus amino acid sequence for the membrane proximal region (MPR) of gp41 of HIV-1. An X represents specific amino acids where alterations can be tolerated.

SEQ ID NO:2 is a consensus amino acid sequence based on each clade consensus sequence of the MPR region from HIV-1.

SEQ ID NO:3 is the ancestral amino acid sequence of the MPR region from HIV-1 clade M. This sequence is also the consensus amino acid sequence of the MPR region from HIV-1 clade AG.

SEQ ID NO:4 is the consensus amino acid sequence of the MPR region from HIV-1 clade A1. This sequence is also the ancestral amino acid sequence of the MPR region from HIV-1 clade A1.

SEQ ID NO:5 is the consensus amino acid sequence of the MPR region from HIV-1 clade A2.

SEQ ID NO:6 is the consensus amino acid sequence of the MPR region from HIV-1 clade B. This sequence is also the ancestral amino acid sequence of the MPR region from HIV-1 clade B.

SEQ ID NO:7 is the consensus amino acid sequence of the MPR region from HIV-1 clade C.

SEQ ID NO:8 is the ancestral amino acid sequence of the MPR region from HIV-1 clade C.

SEQ ID NO:9 is the consensus amino acid sequence of the MPR region from HIV-1 clade D.

SEQ ID NO:10 is the consensus amino acid sequence of the MPR region from HIV-1 clade F1.

SEQ ID NO:11 is the consensus amino acid sequence of the MPR region from HIV-1 clade F2.

SEQ ID NO:12 is the consensus amino acid sequence of the MPR region from HIV-1 clade G.

SEQ ID NO:13 is the consensus amino acid sequence of the MPR region from HIV-1 clade H.

SEQ ID NO:14 is the consensus amino acid sequence of the MPR region from HIV-1 clade AE.

SEQ ID NO:15 is the consensus amino acid sequence of the MPR region from HIV-1 clade AB.

SEQ ID NO:16 is the consensus amino acid sequence of the MPR region from HIV-1 clade 04CPX.

SEQ ID NO:17 is the consensus amino acid sequence of the MPR region from HIV-1 clade 06CPX.

SEQ ID NO:18 is the consensus amino acid sequence of the MPR region from HIV-1 clade 08BC.

SEQ ID NO:19 is the consensus amino acid sequence of the MPR region from HIV-1 clade 10CD.

SEQ ID NO:20 is the consensus amino acid sequence of the MPR region from HIV-1 clade 11CPX.

SEQ ID NO:21 is the consensus amino acid sequence of the MPR region from HIV-1 clade 12BF.

SEQ ID NO:22 is the consensus amino acid sequence of the MPR region from HIV-1 clade 14BG.

SEQ ID NO:23 is the consensus amino acid sequence of the 2F5 epitope.

SEQ ID NO:24 is an amino acid sequence of the 2F5 epitope.

SEQ ID NO:25 is an amino acid sequence for gp160. This sequence is provided as Genbank Accession No. CAD10143, as available on Feb. 14, 2006.

SEQ ID NO:26 is an example of a hydrophobic five residue amino acid sequence.

SEQ ID NO:27 is an example of a hydrophobic ten residue amino acid sequence.

SEQ ID NO:28 is an example of a hydrophobic fifteen residue amino acid sequence.

SEQ ID NO:29 is an example of a hydrophobic twenty-two residue amino acid sequence.

SEQ ID NO:30 is a nucleotide sequence of the HBsAg.

SEQ ID NO:31 is an amino acid sequence of the HBsAg.

SEQ ID NO:32 is an example of a nucleotide sequence for a T helper cell epitope.

SEQ ID NO:33 is an example of an amino acid sequence for a T helper cell epitope.

SEQ ID NO:34 is the CAAX amino acid sequence, where C is cystein, A is an aliphatic amino acid and X is any amino acid.

SEQ ID NO:35 is the core amino acid sequence of the 2F5 epitope.

SEQ ID NO:36 is the core amino acid sequence of the 4E10 epitope.

SEQ ID NO:37 is the linker sequence GPGP.

SEQ ID NO:38 is a forward primer for amplification of the HBsAg.

SEQ ID NO:39 is a reverse primer for amplification of the HBsAg.

SEQ ID NO:40 is the amino acid sequence of a peptide used in the competition ELISA.

SEQ ID NO:41 is a forward primer for amplification of MPR.

SEQ ID NO:42 is a reverse primer for amplification of MPR.

SEQ ID NO:43 is a reverse primer for amplification of MPR-Foldon.

SEQ ID NO:44 is a forward primer for amplification of C-heptad.

SEQ ID NO:45 is a reverse primer for amplification of MPR-Tm5.

SEQ ID NO:46 is a reverse primer for amplification of MPR-Tm10.

SEQ ID NO:47 is a reverse primer for amplification of MPR-Tm15.

SEQ ID NO:48 is a reverse primer for amplification of MPR-Tm23.

SEQ ID NO:49 is a forward primer for amplification of the MPR region with AgeI.

SEQ ID NO:50 is a reverse primer for amplification of the MPR region with AgeI.

SEQ ID NO:51 is a forward primer for amplification of the MPR region with AgeI.

SEQ ID NO:52 is a reverse primer for amplification of the MPR region with AgeI.

SEQ ID NO:53 is a forward primer for amplification of the MPR region with HBsAg (MPRSAG or MPR-N-term).

SEQ ID NO:54 is a reverse primer for amplification of the MPR region with HBsAg (MPRSAG or MPR-N-term).

SEQ ID NO:55 is a forward primer for amplification of SAGMPR-R1 (HBsAg at the N-terminus of MPR).

SEQ ID NO:56 is a reverse primer for amplification of SAGMPR-R1 (HBsAg at the N-terminus of MPR).

SEQ ID NO:57 is an example of a group of five basic amino acid residues.

SEQ ID NO:58 is an example of a group of 10 basic amino acid residues.

SEQ ID NO:59 is a reverse primer for amplification of the HBsAg.

SEQ ID NO:60 is the nucleotide sequence of the CMV/R-HBsAg-C-heptad-MPR-FL construct.

SEQ ID NO:61 is the nucleotide sequence of the CMV/R-MCS-HBsAg125-MPR-128 construct.

SEQ ID NO:62 is the nucleotide sequence of the CMV/R-MCS-HBsAg-MPR construct.

SEQ ID NO:63 is the nucleotide sequence of the CMV/R-MCS-HBsAg-MPR10 construct.

SEQ ID NO:64 is the nucleotide sequence of the CMV/R-MCS-HBsAg-MPR-Tm-C9 construct.

SEQ ID NO:65 is the nucleotide sequence of the CMV/R-MCS-MPR-HBsAg construct.

SEQ ID NO:66 is the nucleotide sequence of the CMV/R-HBsAg-MPR-FL construct.

SEQ ID NO:67 is the nucleotide sequence of the CMV/R-MCS-HBsAg-C-heptad-MPR construct.

SEQ ID NO:68 is the nucleotide sequence of the CMV/R-MCS-HBsAg-MPR5 construct.

SEQ ID NO:69 is the nucleotide sequence of the CMV/R-MCS-HBsAg-MPR15 construct.

SEQ ID NO:70 is the nucleotide sequence of the CMV/R-MCS-HBsAg-STOP construct.

### BRIEF DESCRIPTION OF THE DRAWINGS

FIGS. 1A and B are schematic representations of the constructs developed using hepatitis B surface antigen as a carrier molecule. The gp41 region as shown was cloned at the C-terminus of HBsAg molecule (2-226 aa). The gp41 region from just after the C-heptad repeat to the lysine 683 immediately upstream of the transmembrane was used in two of the constructs. The other two constructs harbor only the MPR region. A foldon trimerization domain was also introduced.

FIG. 2A, B, and C are diagrams of the various constructs. FIG. 2A is a schematic diagram of constructs where various lengths of the transmembrane region were cloned at the C-terminus of MPR to improve the 4E10 recognition. FIG. 2B is a schematic diagram of constructs wherein the MPR was cloned at the N-terminus of the HBsAg (also termed MPRSAG). FIG. 2C is a schematic diagram of constructs that contains the MPR cloned in the hydrophilic immunodominant extra-cellular loop of HBsAg.

FIG. 3A, B, C, D, and E show biochemical analysis of HBsAg-MPR and MPR variants. FIG. 3A shows a graph displaying viral-like particle production by the MPR, MPR-F1, C-heptad MPR, and C-heptad MPR-F1 constructs. FIG. 3B shows a graph displaying viral-like particle production by the MPR-Tm5 (also labeled MPR-5), MPR-Tm10 (also termed MPR-10), MPR-Tm15 (also termed MPR-15), and MPRSAG (also termed MPR-N-term) constructs. FIG. 3C shows a digital image of an SDS gel with partially purified HBsAg-MPR particles from HEK293T cells (lane 4) compared to yeast purified HBsAg particles (lanes 2 and 3). FIG. 3D shows a digital image of Western blot analysis of supernatant purified HBsAg-MPR and MPR variant particles. Lane 1(100 ng) and 2 (50 ng) HBsAg from yeast; lane 3 is MPR-22-C9; lane 4 is MPR-15; lane 5 is MPR-10; Lane 6 is MPR-5; lane 7 is MPR-FL; lanes 8 and 9 are HBsAg-MPR particles from two different batches; lane 10 is marker. FIG. 3E shows a digital image of Western blot analysis of the cell lysate of purified HBsAg-MPR and MPR variants. HBsAg-MPR particles from supernatant were used as control in lane 1. Lane 2 is marker, and particles from the cell lysate are represented in all the other lanes. Lane 3 is HBsAg-MPR; lane 4 is MPR-5; lane 5 is MPR-10; lane 6 is MPR-15; lane 7 is MPR-N-term; lane 8 is yeast purified HBsAg (50 ng).

FIG. 4 is a digital image of an electron micrograph of the HBsAg-C-term MPR particles in the endoplasmic reticulum of HEK293T cells.

FIGS. 5A and B are graphs showing the relative binding of 2F5 (A) and 4E10 (B) to C-term-MPR-Foldon (⋄), HBsAG-C-terminal (C-term)-MPR(□), C-term-C-heptad MPR-Foldon (Δ), and C-term-C-heptad MPR (x), as determined using a sandwich ELISA binding assay.

FIG. 6 is a graph showing the binding of 2F5 (⋄), 4E10 (□) and HIV-Ig (Δ) to HBsAG-C-term-MPR particles.

FIGS. 7A and B are graphs showing the relative binding of 2F5 (A) and 4E10 (B) to C-term-MPR (x), C-term-MPR-5 (∘), C-term-MPR-10 (□) and C-term-MPR-15 (Δ) particles.

FIG. 8 is a graph showing binding of 2F5 and 4E10 to HBsAG-C-term-MPR, HBsAG-N-term-MPR and HBsAG-L-loop-MPR particles.

FIG. 9 is a graph showing competition of 2F5 binding to HBsAg-MPR particles by a 16-mer peptide harboring the 2F5 epitope. The peptide was serially diluted (0 to 42.5 ug/ml) along with 1 □g/ml of 2F5 Ab (⋄), or human sera #20 (□), #30 (Δ), and #881 (x) (at 1:1000 dilution).

FIG. 10 is a graph showing binding of 2F5 (□) or HIV-1 positive human sera (#1 (Δ), #30 (∘); #5 (⋄); #20 (□)) to the HBsAg with or without the membrane proximal region (MPR), as determined by sandwich ELISA.

FIG. 11 is a graph showing binding of HIV-1 positive human sera (#1(Δ); #5 (x); #20 (*); #28 (∘); (#30 (|); #45 (−)) to the HBsAg using a sandwich ELISA.

FIGS. 12A and B are graphs showing the effect of lipid on the binding of 4E10 (A) and 2F5 (B) to the HBsAg-MPR Particles. (∘) Original; (□) No lipid; (Δ) Synthetic lipid DOPC:DOPS 7:3.

FIG. 13A, B, and C are graphs showing analysis of rabbit antisera to 2F5 epitope-KLH. FIG. 13A shows ELISA analysis of rabbit sera (rabbit A (□); rabbit B (Δ)) binding to 2F5 peptide; 2F5 (x); preimmune sera (Δ). FIG. 13B shows binding of rabbit A sera (□) to cell-surface ADAgp160; preimmune sera (Δ). FIG. 13C shows binding of 2F5 to cell surface gp160.

FIG. 14A, B, C, and D are graphs showing analysis of guinea pig antisera to HBsAg-MPR particles. FIG. 14A shows the titer of antibody binding to surface antigen. FIG. 14B shows binding of preimmune (H1 (⋄) or H4(+)) and immune (H1(□) or H4(−)) sera to cell surface-expressed ADA gp160. FIG. 14C shows binding of sera (H1(□) and H4(−) to the 2F5 epitope). FIG. 14D shows the binding of preimmune (H1(⋄) or H4(+)) and immune (H1 (□) or H4(−)) sera to MPR.

FIGS. 15A and B are graphs showing cell surface binding of antisera elicited by HBsAg-MPR particles. FIG. 15A shows binding to MPR expressed on the cell surface by HBsAg-C-term-MPR immune sera versus preimmune sera and HBsAg control sera. FIG. 15B shows binding to JR-FL gp160 by HBsAg-C-term-MPR immune sera versus preimmune sera and HBsAg control sera.

FIGS. 16A and B are graphs showing the selection of K562 cells that display antibodies to either HBsAG or to the MPR region (2F5 and 4E10). FIG. 16A shows selection by NF5 (⋄) as compared to HIV-Ig (□). FIG. 16B shows selection by 2F5 (⋄) or 4E10 (□) as compared to HIV-Ig (Δ).

FIGS. 17A and B are diagrams of the CMV/R-HBsAg-C-heptad-MPR-FL (A) and the CMV/R-MCS-HBsAg125-MPR-128 (B) constructs.

FIGS. 18A and B are diagrams of the CMV/R-MCS-HBsAg-MPR (A) and the CMV/R-MCS-HBsAg-MPR10 (B) constructs.

FIGS. 19A and B are diagrams of the CMV/R-MCS-HBsAg-MPR-Tm-C9 (A) and the CMV/R-MCS-MPR-HBsAg (B) constructs.

FIGS. 20A and B are diagrams of the CMV/R-HBsAg-MPR-FL (A) and CMV/R-MCS-HBsAg-C-heptad-MPR (B) constructs.

FIGS. 21A and B are diagrams of the CMV/R-MCS-HBsAg-MPR5 (A) and CMV/R-MCS-HBsAg-MPR15 (B) constructs.

FIG. 22 is a diagram of the CMV/R-MCS-HBsAg-STOP construct.

### DETAILED DESCRIPTION

Historically, viral vaccines have been live-attenuated or chemically inactivated forms of the virus. However, this approach has limited utility when used for human immunodeficiency virus. Recombinant HBsAg-gp120 has been used to present approximately amino acids 1-500 of gp120. However, the presentation of gp120 in this form has not successfully been used to produce neutralizing antibodies. Disclosed herein is the use of the immunogenic Hepatitis B surface antigen (HBsAg) particulate platform to array epitopes from the conserved, neutralization-sensitive membrane proximal region (MPR) of HIV-1, and the use of this platform to induce an immune response to HIV-1 using specific antigenic epitopes of gp41. Specifically, it is disclosed herein that the HBsAg can be used as a carrier for a multi-array presentation of the antigenic components of the HIV envelope protein (env), such as to induce an immune response to highly conserved, hydrophobic 2F5 and 4E10 neutralizing determinants from gp41. In addition, the use of the HBsAg platform allows presentation of the MPR as an immunogen in an appropriate lipid context. Viral B-cell epitopes that are presented in rigid, highly repetitive, paracrystalline forms can induce neutralizing antibodies that help to clear virus. Furthermore, the arrayed B-cell epitopes can be recognized as foreign and induce B-cell activation to produce protective neutralizing antibodies against surface antigens.

### Description of Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Adjuvant: A vehicle used to enhance antigenicity; such as a suspension of minerals (alum, aluminum hydroxide, or phosphate) on which antigen is adsorbed; or water-in-oil emulsion in which antigen solution is emulsified in mineral oil (Freund incomplete adjuvant), sometimes with the inclusion of killed mycobacteria (Freund's complete adjuvant) to further enhance antigenicity (inhibits degradation of antigen and/or causes influx of macrophages). Immunstimulatory oligonucleotides (such as those including a CpG motif) can also be used as adjuvants (for example see U.S. Pat. No. 6,194,388; U.S. Pat. No. 6,207,646; U.S. Pat. No. 6,214,806; U.S. Pat. No. 6,218,371; U.S. Pat. No. 6,239,116; U.S. Pat. No. 6,339,068; U.S. Pat. No. 6,406,705; and U.S. Pat. No. 6,429,199).

Antigen: A compound, composition, or substance that can stimulate the production of antibodies or a T cell response in an animal, including compositions that are injected or absorbed into an animal. An antigen reacts with the products of specific humoral or cellular immunity, including those induced by heterologous immunogens. The term is used interchangeably with the term “immunogen.” The term “antigen” includes all related antigenic epitopes. An “antigenic polypeptide” is a polypeptide to which an immune response, such as a T cell response or an antibody response, can be stimulated. “Epitope” or “antigenic determinant” refers to a site on an antigen to which B and/or T cells respond. In one embodiment, T cells respond to the epitope when the epitope is presented in conjunction with an MHC molecule. Epitopes can be formed both from contiguous amino acids (linear) or noncontiguous amino acids juxtaposed by tertiary folding of an antigenic polypeptide (conformational). Epitopes formed from contiguous amino acids are typically retained on exposure to denaturing solvents whereas epitopes formed by tertiary folding are typically lost on treatment with denaturing solvents. Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 5 and 15 amino acids, such as, 9, 10, 12 or 15 amino acids. The amino acids are in a unique spatial conformation. Methods of determining spatial conformation of epitopes include, for example, x-ray crystallography and multi-dimensional nuclear magnetic resonance spectroscopy. The term “antigen” denotes both subunit antigens, (for example, antigens which are separate and discrete from a whole organism with which the antigen is associated in nature), as well as killed, attenuated or inactivated bacteria, viruses, fungi, parasites or other microbes. Antibodies such as anti-idiotype antibodies, or fragments thereof, and synthetic peptide mimotopes, which can mimic an antigen or antigenic determinant, are also captured under the definition of antigen as used herein. Similarly, an oligonucleotide or polynucleotide which expresses an antigen or antigenic determinant in vivo, such as in gene therapy and DNA immunization applications, is also included in the definition of antigen herein.

An “antigen,” when referring to a protein, includes a protein with modifications, such as deletions, additions and substitutions (generally conservative in nature) to the native sequence, so long as the protein maintains the ability to elicit an immunological response, as defined herein. These modifications may be deliberate, as through site-directed mutagenesis, or may be accidental, such as through mutations of hosts which produce the antigens.

Antigen Delivery Platform or Epitope Mounting Platform: In the context of the present disclosure, the terms “antigen delivery platform” and “epitope mounting platform” refer to a macromolecular complex including one or more antigenic epitopes. Delivery of an antigen (including one or more epitopes) in the context of an epitope mounting platform enhances, increases, ameliorates or otherwise improves a desired antigen-specific immune response to the antigenic epitope(s). The molecular constituents of the antigen delivery platform may be antigenically neutral or may be immunologically active, that is, capable of generating a specific immune response. Nonetheless, the term antigen delivery platform is utilized to indicate that a desired immune response is generated against a selected antigen that is a component of the macromolecular complex other than the platform polypeptide to which the antigen is attached. Accordingly, the epitope mounting platform is useful for delivering a wide variety of antigenic epitopes, including antigenic epitopes of pathogenic organisms such as bacteria and viruses. The antigen delivery platform of the present disclosure is particularly useful for the delivery of complex peptide or polypeptide antigens, which may include one or many distinct epitopes.

Amplification: Of a nucleic acid molecule (e.g., a DNA or RNA molecule) refers to use of a technique that increases the number of copies of a nucleic acid molecule in a specimen. An example of amplification is the polymerase chain reaction (PCR), in which a biological sample collected from a subject is contacted with a pair of oligonucleotide primers, under conditions that allow for the hybridization of the primers to a nucleic acid template in the sample. The primers are extended under suitable conditions, dissociated from the template, and then re-annealed, extended, and dissociated to amplify the number of copies of the nucleic acid. The product of amplification may be characterized by electrophoresis, restriction endonuclease cleavage patterns, oligonucleotide hybridization or ligation, and/or nucleic acid sequencing using standard techniques. Other examples of amplification include strand displacement amplification, as disclosed in U.S. Pat. No. 5,744,311; transcription-free isothermal amplification, as disclosed in U.S. Pat. No. 6,033,881; repair chain reaction amplification, as disclosed in WO 90/01069; ligase chain reaction amplification, as disclosed in EP-A-320 308; gap filling ligase chain reaction amplification, as disclosed in U.S. Pat. No. 5,427,930; and NASBA™ RNA transcription-free amplification, as disclosed in U.S. Pat. No. 6,025,134.

Antibody: Immunoglobulin molecules and immunologically active portions of immunoglobulin molecules, that is, molecules that contain an antigen binding site that specifically binds (immunoreacts with) an antigen.

A naturally occurring antibody (e.g., IgG, IgM, IgD) includes four polypeptide chains, two heavy (H) chains and two light (L) chains interconnected by disulfide bonds. However, it has been shown that the antigen-binding function of an antibody can be performed by fragments of a naturally occurring antibody. Thus, these antigen-binding fragments are also intended to be designated by the term “antibody.” Specific, non-limiting examples of binding fragments encompassed within the term antibody include (i) a Fab fragment consisting of the V_{L}, V_{H}, C_{L} and C_{H1} domains; (ii) an F_{d} fragment consisting of the V_{H} and C_{H1} domains; (iii) an Fv fragment consisting of the V_{L} and V_{H} domains of a single arm of an antibody, (iv) a dAb fragment (Ward et al., Nature 341:544-546, 1989) which consists of a V_{H} domain; (v) an isolated complimentarity determining region (CDR); and (vi) a F(ab′)₂ fragment, a bivalent fragment comprising two Fab fragments linked by a disulfide bridge at the hinge region.

Methods of producing polyclonal and monoclonal antibodies are known to those of ordinary skill in the art, and many antibodies are available. See, e.g., Coligan, Current Protocols in Immunology Wiley/Greene, NY, 1991; and Harlow and Lane, Antibodies: A Laboratory Manual Cold Spring Harbor Press, NY, 1989; Stites et al., (eds.) Basic and Clinical Immunology (4th ed.) Lange Medical Publications, Los Altos, Calif., and references cited therein; Goding, Monoclonal Antibodies: Principles and Practice (2d ed.) Academic Press, New York, N.Y. 1986; and Kohler and Milstein, Nature 256: 495-497, 1975. Other suitable techniques for antibody preparation include selection of libraries of recombinant antibodies in phage or similar vectors. See, Huse et al., Science 246: 1275-1281, 1989; and Ward et al., Nature 341: 544-546, 1989. “Specific” monoclonal and polyclonal antibodies and antisera (or antiserum) will usually bind with a K_{D} of at least about 0.1 μM, preferably at least about 0.01 μM or better, and most typically and preferably, 0.001 μM or better.

Immunoglobulins and certain variants thereof are known and many have been prepared in recombinant cell culture (e.g., see U.S. Pat. No. 4,745,055; U.S. Pat. No. 4,444,487; WO 88/03565; EP 256,654; EP 120,694; EP 125,023; Faoulkner et al., Nature 298:286, 1982; Morrison, J. Immunol. 123:793, 1979; Morrison et al., Ann Rev. Immunol 2:239, 1984). Detailed methods for preparation of chimeric (humanized) antibodies can be found in U.S. Pat. No. 5,482,856. Additional details on humanization and other antibody production and engineering techniques can be found in Borrebaeck (ed), Antibody Engineering, 2^{nd} Edition Freeman and Company, NY, 1995; McCafferty et al., Antibody Engineering, A Practical Approach, IRL at Oxford Press, Oxford, England, 1996, and Paul Antibody Engineering Protocols Humana Press, Towata, N.J., 1995.

Animal: Living multi-cellular vertebrate organisms, a category that includes, for example, mammals and birds. The term mammal includes both human and non-human mammals. Similarly, the term “subject” includes both human and veterinary subjects.

Conservative variants: “Conservative” amino acid substitutions are those substitutions that do not substantially affect or decrease a desired activity of a protein or polypeptide. For example, in the context of the present disclosure, a conservative amino acid substitution does not substantially alter or decrease the immunogenicity of an antigenic epitope. Similarly, a conservative amino acid substitution does not substantially affect the structure or, for example, the stability of a protein or polypeptide. Specific, non-limiting examples of a conservative substitution include the following examples:

| Original Residue | Conservative Substitutions |
|---|---|
| Ala | Ser |
| Arg | Lys |
| Asn | Gln; His |
| Asp | Glu |
| Cys | Ser |
| Gln | Asn |
| Glu | Asp |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

The term conservative variation also includes the use of a substituted amino acid in place of an unsubstituted parent amino acid, provided that antibodies raised to the substituted polypeptide also immunoreact with the unsubstituted polypeptide. Non-conservative substitutions are those that reduce an activity or antigenicity or substantially alter a structure, such as a secondary or tertiary structure, of a protein or polypeptide.

cDNA (complementary DNA): A piece of DNA lacking internal, non-coding segments (introns) and regulatory sequences that determine transcription. cDNA is typically synthesized in the laboratory by reverse transcription from messenger RNA extracted from cells.

Diagnostic: Identifying the presence or nature of a pathologic condition, such as, but not limited to a condition induced by a viral or other pathogen. Diagnostic methods differ in their sensitivity and specificity. The “sensitivity” of a diagnostic assay is the percentage of diseased individuals who test positive (percent of true positives). The “specificity” of a diagnostic assay is 1 minus the false positive rate, where the false positive rate is defined as the proportion of those without the disease who test positive. While a particular diagnostic method may not provide a definitive diagnosis of a condition, it suffices if the method provides a positive indication that aids in diagnosis. “Prognostic” is the probability of development (or for example, the probability of severity) of a pathologic condition, such as a symptom induced by a viral infection or other pathogenic organism, or resulting indirectly from such an infection.

Epitope: An antigenic determinant. These are particular chemical groups or peptide sequences on a molecule that are antigenic, that is, that elicit a specific immune response. An antibody specifically binds a particular antigenic epitope on a polypeptide. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5, about 9, or 8-10 amino acids in a unique spatial conformation. Methods of determining spatial conformation of epitopes include, for example, x-ray crystallography and multi-dimensional nuclear magnetic resonance spectroscopy. See, e.g., “Epitope Mapping Protocols” in Methods in Molecular Biology, Vol. 66, Glenn E. Morris, Ed (1996). In one embodiment, an epitope binds an MHC molecule, e.g., an HLA molecule or a DR molecule. These molecules bind polypeptides having the correct anchor amino acids separated by about eight or nine amino acids

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcription terminators, a start codon (typically, ATG) in front of a protein-encoding gene, splicing signal for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences.

Promoter: A promoter is a minimal sequence sufficient to direct transcription. Also included are those promoter elements which are sufficient to render promoter-dependent gene expression controllable for cell-type specific, tissue-specific, or inducible by external signals or agents; such elements may be located in the 5′ or 3′ regions of the gene. Both constitutive and inducible promoters are included (see e.g., Bitter et al., Methods in Enzymology 153:516-544, 1987). For example, when cloning in bacterial systems, inducible promoters such as pL of bacteriophage lambda, plac, ptrp, ptac (ptrp-lac hybrid promoter) and the like may be used. In one embodiment, when cloning in mammalian cell systems, promoters derived from the genome of mammalian cells (for example, metallothionein promoter) or from mammalian viruses (for example, the retrovirus long terminal repeat; the adenovirus late promoter; the vaccinia virus 7.5K promoter) can be used. Promoters produced by recombinant DNA or synthetic techniques may also be used to provide for transcription of the nucleic acid sequences.

Hepatitis B Surface Antigen (HBsAg): HBsAg is composed of 3 polypeptides, preS1, preS2 and S that are produced from alternative translation start sites. The surface proteins have many functions, including attachment and penetration of the virus into hepatocytes at the beginning of the infection process. The surface antigen is a principal component of the hepatitis B envelope.

Host cells: Cells in which a polynucleotide, for example, a polynucleotide vector or a viral vector, can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Human Immunodeficiency Virus: A virus, known to cause AIDS, that includes HIV-1 and HIV-2. HIV-1 is composed of two copies of single-stranded RNA enclosed by a conical capsid including the viral protein p24, typical of lentiviruses. The capsid is surrounded by a plasma membrane of host-cell origin.

The envelope protein of HIV-1 is made up of a glycoprotein called gp160. The mature, virion associated envelope protein is a trimeric molecule composed of three gp120 and three gp41 subunits held together by weak noncovalent interactions. This structure is highly flexible and undergoes substantial conformational changes upon gp120 binding with CD4 and chemokine coreceptors, which leads to exposure of the fusion peptides of gp41 that insert into the target cell membrane and mediate viral entry. Following oligomerization in the endoplasmic reticulum, the gp160 precursor protein is cleaved by cellular proteases and is transported to the cell surface. During the course of HIV-1 infection, the gp120 and gp41 subunits are shed from virions and virus-infected cells due to the noncovalent interactions between gp120 and gp41 and between gp41 subunits. The membrane proximal region (MPR) is approximately the 30 amino acids immediately upstream of the transmembrane region of gp41. The MPR is highly hydrophobic (50% of residues are hydrophobic) and is highly conserved across many HIV clades (Zwick, M. B., et al., J Virol, 2001. 75 (22): p. 10892-905). The conserved membrane-proximal region (MPR) of HIV-1 gp41 is a target of two broadly neutralizing human monoclonal antibodies, 2F5 and 4E10. The core of the 2F5 epitope has been shown to be ELDKWAS (SEQ ID NO:35). With this epitope, the residues D, K, and W were found to be most critical for recognition by 2F5. The core of the 4E10 epitope, NWFDIT (SEQ ID NO:36), maps just C-terminal to the 2F5 epitope on the gp41 ectodomain.

Immune response: A response of a cell of the immune system, such as a B cell, T cell, or monocyte, to a stimulus. In some cases, the response is specific for a particular antigen (that is, an “antigen-specific response”). In some cases, an immune response is a T cell response, such as a CD4+ response or a CD8+ response. Alternatively, the response is a B cell response, and results in the production of specific antibodies. For purposes of the present invention, a “humoral immune response” refers to an immune response mediated by antibody molecules, while a “cellular immune response” is one mediated by T-lymphocytes and/or other white blood cells. A “protective immune response” is an immune response that inhibits a detrimental function or activity (such as a detrimental effect of a pathogenic organism such as a virus), reduces infection by a pathogenic organism (such as, a virus), or decreases symptoms that result from infection by the pathogenic organism. A protective immune response can be measured, for example, by the inhibition of viral replication or plaque formation in a plaque reduction assay or ELISA-neutralization assay (NELISA), or by measuring resistance to viral challenge in vivo.

An immunogenic composition can induce a B cell response. The ability of a particular antigen to stimulate a B cell response can be measured by determining if antibodies are present that bind the antigen. In one example, neutralizing antibodies are produced.

One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surface of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells.

The ability of a particular antigen to stimulate a cell-mediated immunological response may be determined by a number of assays, such as by lymphoproliferation (lymphocyte activation) assays, CTL cytotoxic cell assays, or by assaying for T-lymphocytes specific for the antigen in a sensitized subject. Such assays are well known in the art. See, for example, Erickson et al. (1993) J. Immunol. 151:4189-4199; Doe et al. (1994) Eur. J. Immunol. 24:2369-2376. Recent methods of measuring cell-mediated immune response include measurement of intracellular cytokines or cytokine secretion by T-cell populations, or by measurement of epitope specific T-cells (for example, by the tetramer technique) (reviewed by McMichael and O'Callaghan (1998) J. Exp. Med. 187(9)1367-1371; Mcheyzer-Williams et al. (1996) Immunol. Rev. 150:5-21; Lalvani et al. (1997)J. Exp. Med. 186:859-865).

Thus, an immunological response as used herein may be one which stimulates the production of CTLs, and/or the production or activation of helper T-cells. The antigen of interest may also elicit an antibody-mediated immune response. Hence, an immunological response may include one or more of the following effects: the production of antibodies by B-cells; and/or the activation of suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

Immunogenic peptide: A peptide which comprises an allele-specific motif or other sequence such that the peptide will bind an MHC molecule and induce a cytotoxic T lymphocyte (“CTL”) response, or a B cell response (e.g. antibody production) against the antigen from which the immunogenic peptide is derived.

Immunogenic composition: A composition comprising at least one epitope of a virus, or other pathogenic organism, that induces a measurable CTL response, or induces a measurable B cell response (for example, production of antibodies that specifically bind the epitope). It further refers to isolated nucleic acids encoding an immunogenic epitope of virus or other pathogen that can be used to express the epitope (and thus be used to elicit an immune response against this polypeptide or a related polypeptide expressed by the pathogen). For in vitro use, the immunogenic composition may consist of the isolated nucleic acid, protein or peptide. For in vivo use, the immunogenic composition will typically include the nucleic acid, protein or peptide in pharmaceutically acceptable carriers or excipients, and/or other agents, for example, adjuvants. An immunogenic polypeptide (such as an antigenic polypeptide), or nucleic acid encoding the polypeptide, can be readily tested for its ability to induce a CTL or antibody response by art-recognized assays.

Isolated: An “isolated” biological component (such as a nucleic acid or protein or organelle) has been substantially separated or purified away from other biological components in the cell of the organism in which the component naturally occurs, for example, other chromosomal and extra-chromosomal DNA and RNA, proteins and organelles. Nucleic acids and proteins that have been “isolated” include nucleic acids and proteins purified by standard purification methods. The term also embraces nucleic acids and proteins prepared by recombinant expression in a host cell as well as chemically synthesized nucleic acids.

Label: A detectable compound or composition that is conjugated directly or indirectly to another molecule to facilitate detection of that molecule. Specific, non-limiting examples of labels include fluorescent tags, affinity tags, enzymatic linkages, and radioactive isotopes. An affinity tag is a peptide or polypeptide sequence capable of specifically binding to a specified substrate, for example, an organic, non-organic or enzymatic substrate or cofactor. A polypeptide including a peptide or polypeptide affinity tag can typically be recovered, for example, purified or isolated, by means of the specific interaction between the affinity tag and its substrate. An exemplary affinity tag is a poly-histidine (e.g., six-histidine) affinity tag which can specifically bind to non-organic metals such as nickel and/or cobalt. Additional affinity tags are well known in the art.

Linking peptide: A linking peptide (or linker sequence) is an amino acid sequence that covalently links two polypeptide domains. Linking peptides can be included between the rotavirus NSP2 polypeptide and an antigenic epitope to provide rotational freedom to the linked polypeptide domains and thereby to promote proper domain folding. Linking peptides, which are generally between 2 and 25 amino acids in length, are well known in the art and include, but are not limited to the amino acid sequences glycine-proline-glycine-proline (GPGP) (SEQ ID NO:37) and glycine-glycine-serine (GGS), as well as the glycine(4)-serine spacer described by Chaudhary et al., Nature 339:394-397, 1989. In some cases multiple repeats of a linking peptide are present.

Lymphocytes: A type of white blood cell that is involved in the immune defenses of the body. There are two main types of lymphocytes: B cells and T cells. “T lymphocytes” or “T cells” are non-antibody producing lymphocytes that constitute a part of the cell-mediated arm of the immune system. T cells arise from immature lymphocytes that migrate from the bone marrow to the thymus, where they undergo a maturation process under the direction of thymic hormones. Here, the mature lymphocytes rapidly divide increasing to very large numbers. The maturing T cells become immunocompetent based on their ability to recognize and bind a specific antigen. Activation of immunocompetent T cells is triggered when an antigen binds to the lymphocyte's surface receptors. T cells include, but are not limited to, CD4⁺ T cells and CD8⁺ T cells. A CD4⁺ T lymphocyte is an immune cell that carries a marker on its surface known as “cluster of differentiation 4” (CD4). These cells, also known as helper T cells, help orchestrate the immune response, including antibody responses as well as killer T cell responses. CD8⁺ T cells carry the “cluster of differentiation 8” (CD8) marker. In one embodiment, a CD8 T cell is a cytotoxic T lymphocyte. In another embodiment, a CD8 cell is a suppressor T cell.

Mammal: This term includes both human and non-human mammals unless otherwise specified. Similarly, the term “subject” includes both human and veterinary subjects.

Oligonucleotide: A linear polynucleotide sequence of up to about 100 nucleotide bases in length.

Open reading frame (“ORF”): A series of nucleotide triplets (codons) coding for amino acids without any internal termination codons. These sequences are usually translatable into a polypeptide (peptide or protein).

Operatively linked: A first nucleic acid sequence is operatively linked with a second nucleic acid sequence when the first nucleic acid sequence is placed in a functional relationship with the second nucleic acid sequence. For instance, a promoter is operatively linked to a coding sequence if the promoter affects the transcription or expression of the coding sequence. Generally, operatively linked DNA sequences are contiguous and, where necessary to join two protein-coding regions, in the same reading frame, for example, two polypeptide domains or components of a fusion protein.

Pharmaceutically acceptable carriers and/or pharmaceutically acceptable excipients: The pharmaceutically acceptable carriers or excipients of use are conventional. Remington's Pharmaceutical Sciences, by E. W. Martin, Mack Publishing Co., Easton, Pa., 15th Edition (1975), describes compositions and formulations suitable for pharmaceutical delivery of the polypeptides and polynucleotides disclosed herein.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

A “therapeutically effective amount” is a quantity of a composition used to achieve a desired effect in a subject. For instance, this can be the amount of the composition necessary to inhibit viral (or other pathogen) replication or to prevent or measurably alter outward symptoms of viral (or other pathogenic) infection. When administered to a subject, a dosage will generally be used that will achieve target tissue concentrations (for example, in lymphocytes) that has been shown to achieve an in vitro effect.

Polynucleotide: The term polynucleotide or nucleic acid sequence refers to a polymeric form of nucleotide at least 10 bases in length. A recombinant polynucleotide includes a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single- and double-stranded forms of DNA.

Polypeptide: Any chain of amino acids, regardless of length or post-translational modification (for example, glycosylation or phosphorylation), such as a protein or a fragment or subsequence of a protein. The term “peptide” is typically used to refer to a chain of amino acids of between 3 and 30 amino acids in length. For example an immunologically relevant peptide may be between about 7 and about 25 amino acids in length, e.g., between about 8 and about 10 amino acids.

In the context of the present disclosure, a polypeptide can be a fusion protein comprising a plurality of constituent polypeptide (or peptide) elements. Typically, the constituents of the fusion protein are genetically distinct, that is, they originate from distinct genetic elements, such as genetic elements of different organisms or from different genetic elements (genomic components) or from different locations on a single genetic element, or in a different relationship than found in their natural environment. Nonetheless, in the context of a fusion protein the distinct elements are translated as a single polypeptide. The term monomeric fusion protein (or monomeric fusion protein subunit) is used synonymously with such a single fusion protein polypeptide to clarify reference to a single constituent subunit where the translated fusion proteins assume a multimeric tertiary structure.

Specifically, in an embodiment, a monomeric fusion protein subunit includes in an N-terminal to C-terminal direction: a viral NSP2 polypeptide; a linear linking peptide; and an antigenic polypeptide or epitope translated into a single polypeptide monomer. A plurality (for example, 4, 8, 12 or 16) of monomeric fusion protein subunits self-assembles into a multimeric ring structure.

Preventing or treating a disease: Inhibiting infection by a pathogen such as a virus, such as a rotavirus or other virus, refers to inhibiting the full development of a disease. For example, inhibiting a viral infection refers to lessening symptoms resulting from infection by the virus, such as preventing the development of symptoms in a person who is known to have been exposed to the virus, or to lessening virus number or infectivity of a virus in a subject exposed to the virus. “Treatment” refers to a therapeutic or prophylactic intervention that ameliorates or prevents a sign or symptom of a disease or pathological condition related to infection of a subject with a virus or other pathogen.

Probes and primers: A probe comprises an isolated nucleic acid attached to a detectable label or reporter molecule. Primers are short nucleic acids, preferably DNA oligonucleotides, for example, a nucleotide sequence of about 15 nucleotides or more in length. Primers may be annealed to a complementary target DNA strand by nucleic acid hybridization to form a hybrid between the primer and the target DNA strand, and then extended along the target DNA strand by a DNA polymerase enzyme. Primer pairs can be used for amplification of a nucleic acid sequence, for example, by the polymerase chain reaction (PCR) or other nucleic-acid amplification methods known in the art. One of skill in the art will appreciate that the specificity of a particular probe or primer increases with its length. Thus, for example, a primer comprising 20 consecutive nucleotides will anneal to a target with a higher specificity than a corresponding primer of only about 15 nucleotides. Thus, in order to obtain greater specificity, probes and primers may be selected that comprise 20, 25, 30, 35, 40, 50 or more consecutive nucleotides.

Promoter: A promoter is an array of nucleic acid control sequences that directs transcription of a nucleic acid. A promoter includes necessary nucleic acid sequences near the start site of transcription, such as in the case of a polymerase II type promoter (a TATA element). A promoter also optionally includes distal enhancer or repressor elements which can be located as much as several thousand base pairs from the start site of transcription. Both constitutive and inducible promoters are included (see, e.g., Bitter et al., Methods in Enzymology 153:516-544, 1987).

Specific, non-limiting examples of promoters include promoters derived from the genome of mammalian cells (e.g., metallothionein promoter) or from mammalian viruses (e.g., the retrovirus long terminal repeat; the adenovirus late promoter; the vaccinia virus 7.5K promoter) may be used. Promoters produced by recombinant DNA or synthetic techniques may also be used. A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Protein purification: the fusion polypeptides disclosed herein can be purified (and/or synthesized) by any of the means known in the art (see, e.g., Guide to Protein Purification, ed. Deutscher, Meth. Enzymol. 185, Academic Press, San Diego (1990); and Scopes, Protein Purification: Principles and Practice, Springer Verlag, New York (1982). Substantial purification denotes purification from other proteins or cellular components. A substantially purified protein is at least 60%, 70%, 80%, 90%, 95% or 98% pure. Thus, in one specific, non-limiting example, a substantially purified protein is 90% free of other proteins or cellular components.

Purified: The term “purified” does not require absolute purity; rather, it is intended as a relative term. Thus, for example, a purified nucleic acid is one in which the nucleic acid is more enriched than the nucleic acid in its natural environment within a cell. Similarly, a purified peptide preparation is one in which the peptide or protein is more enriched than the peptide or protein is in its natural environment within a cell. In one embodiment, a preparation is purified such that the protein or peptide represents at least 50% (such as, but not limited to, 70%, 80%, 90%, 95%, 98% or 99%) of the total peptide or protein content of the preparation.

Recombinant: A recombinant nucleic acid is one that has a sequence that is not naturally occurring or has a sequence that is made by an artificial combination of two otherwise separated segments of sequence, for example, a polynucleotide encoding a fusion protein. This artificial combination is often accomplished by chemical synthesis or, more commonly, by the artificial manipulation of isolated segments of nucleic acids, e.g., by genetic engineering techniques.

Sequence identity: The similarity between amino acid (and polynucleotide) sequences is expressed in terms of the similarity between the sequences, otherwise referred to as sequence identity. Sequence identity is frequently measured in terms of percentage identity (or similarity); the higher the percentage, the more similar are the primary structures of the two sequences. In general, the more similar the primary structures of two amino acid sequences, the more similar are the higher order structures resulting from folding and assembly. However, the converse is not necessarily true, and polypeptides with low sequence identity at the amino acid level can nonetheless have highly similar tertiary and quaternary structures. For example, NSP2 homologs with little sequence identity (for example, less than 50% sequence identity, or even less than 30%, or less than 20% sequence identity) share similar higher order structure and assembly properties, such that even distantly related NSP2 proteins assemble into multimeric ring structures as described herein.

Methods of determining sequence identity are well known in the art. Various programs and alignment algorithms are described in: Smith and Waterman, Adv. Appl. Math. 2:482, 1981; Needleman and Wunsch, J. Mol. Biol. 48:443, 1970; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. USA 85:2444, 1988. Altschul et al., Nature Genet. 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet, for use in connection with the sequence analysis programs blastp, blastn, blastx, tblastn and tblastx. A description of how to determine sequence identity using this program is available on the NCBI website on the internet.

Another indicia of sequence similarity between two nucleic acids is the ability to hybridize. The more similar are the sequences of the two nucleic acids, the more stringent the conditions at which they will hybridize. The stringency of hybridization conditions are sequence-dependent and are different under different environmental parameters. Thus, hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ and/or Mg⁺⁺ concentration) of the hybridization buffer will determine the stringency of hybridization, though wash times also influence stringency. Generally, stringent conditions are selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Conditions for nucleic acid hybridization and calculation of stringencies can be found, for example, in Sambrook et al., Molecular Cloning: A Laboratory Manual, Cold Spring Harbor Laboratory Press, Cold Spring Harbor, N.Y., 2001; Tijssen, Hybridization With Nucleic Acid Probes, Part I Theory and Nucleic Acid Preparation, Laboratory Techniques in Biochemistry and Molecular Biology, Elsevier Science Ltd., NY, N.Y., 1993. and Ausubel et al. Short Protocols in Molecular Biology, 4^{th} ed., John Wiley & Sons, Inc., 1999.

For purposes of the present disclosure, “stringent conditions” encompass conditions under which hybridization will only occur if there is less than 25% mismatch between the hybridization molecule and the target sequence. “Stringent conditions” may be broken down into particular levels of stringency for more precise definition. Thus, as used herein, “moderate stringency” conditions are those under which molecules with more than 25% sequence mismatch will not hybridize; conditions of “medium stringency” are those under which molecules with more than 15% mismatch will not hybridize, and conditions of “high stringency” are those under which sequences with more than 10% mismatch will not hybridize. Conditions of “very high stringency” are those under which sequences with more than 6% mismatch will not hybridize. In contrast nucleic acids that hybridize under “low stringency conditions include those with much less sequence identity, or with sequence identify over only short subsequences of the nucleic acid.

For example, a specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y., 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Subject: Living multi-cellular vertebrate organisms, a category that includes both human and veterinary subjects, including human and non-human mammals.

Therapeutically active polypeptide: An agent, such as an epitope of a virus or other pathogen that causes induction of an immune response, as measured by clinical response (for example increase in a population of immune cells, increased cytolytic activity against the epitope). Therapeutically active molecules can also be made from nucleic acids. Examples of a nucleic acid based therapeutically active molecule is a nucleic acid sequence that encodes an epitope of a protein of a virus or other pathogen, wherein the nucleic acid sequence is operatively linked to a control element such as a promoter.

In one embodiment, a therapeutically effective amount of an antigenic epitope is an amount used to generate an immune response, or inhibit a function or activity of a virus or other pathogen. Treatment refers to a therapeutic intervention that ameliorates a sign or symptom resulting from exposure to a virus or other pathogen, or a reduction in viral or pathogen load. Treatment also refers to a prophylactic intervention to prevent a sign or symptom that results from exposure to a virus or other pathogen, or to reduce viral or pathogen load.

Transduced or Transfected: A transduced cell is a cell into which a nucleic acid molecule has been introduced by molecular biology techniques. As used herein, the term introduction or transduction encompasses all techniques by which a nucleic acid molecule might be introduced into such a cell, including transfection with viral vectors, transformation with plasmid vectors, and introduction of naked DNA by electroporation, lipofection, and particle gun acceleration.

Vaccine: A vaccine is a pharmaceutical composition that elicits a prophylactic or therapeutic immune response in a subject. In some cases, the immune response is a protective immune response. Typically, a vaccine elicits an antigen-specific immune response to an antigen of a pathogen, for example, a bacterial or viral pathogen, or to a cellular constituent correlated with a pathological condition. A vaccine may include a polynucleotide, a peptide or polypeptide, a virus, a bacteria, a cell or one or more cellular constituents. In some cases, the virus, bacteria or cell may be inactivated or attenuated to prevent or reduce the likelihood of infection, while maintaining the immunogenicity of the vaccine constituent.

Vector: A nucleic acid molecule as introduced into a host cell, thereby producing a transformed host cell. A vector may include nucleic acid sequences that permit it to replicate in a host cell, such as an origin of replication. A vector may also include one or more selectable marker gene and other genetic elements known in the art.

Virus-like particle or VLP: A nonreplicating, viral shell, derived from any of several viruses. VLPs are generally composed of one or more viral proteins, such as, but not limited to, those proteins referred to as capsid, coat, shell, surface and/or envelope proteins, or particle-forming polypeptides derived from these proteins. VLPs can form spontaneously upon recombinant expression of the protein in an appropriate expression system. Methods for producing particular VLPs are known in the art. The presence of VLPs following recombinant expression of viral proteins can be detected using conventional techniques known in the art, such as by electron microscopy, biophysical characterization, and the like. See, for example, Baker et al. (1991) Biophys. J. 60:1445-1456; Hagensee et al. (1994) J. Virol. 68:4503-4505. For example, VLPs can be isolated by density gradient centrifugation and/or identified by characteristic density banding. Alternatively, cryoelectron microscopy can be performed on vitrified aqueous samples of the VLP preparation in question, and images recorded under appropriate exposure conditions.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety. In case of conflict, the present specification, including explanations of terms, will control. In addition, the materials, methods, and examples are illustrative only and not intended to be limiting.

### Hepatitis B Antigen as a Platform for HIV-1 Epitopes

Historically, viral vaccines have been live-attenuated or chemically inactivated forms of the virus. However, this approach has limited utility when used for human immunodeficiency virus. Disclosed herein is the use of the immunogenic Hepatitis B surface antigen (HBsAg) particulate platform to array epitopes from the conserved, neutralization-sensitive membrane proximal region (MPR) of HIV-1, and the use of this platform to induce an immune response to HIV-1.

Recombinant HBsAg-gp120 previously has been used to present approximately amino acids 1-500 gp120. However, presentation of gp120 in this form has not successfully been used to produce neutralizing antibodies. It is disclosed herein that HBsAg can be used as a carrier for a multi-array presentation of antigenic components of the HIV envelope protein (env), such as to induce an immune response to highly conserved, hydrophobic 2F5 and 4E10 neutralizing determinants. It is shown herein that viral B-cell epitopes that are presented in rigid, highly repetitive, paracrystalline forms can induce neutralizing antibodies that help to clear virus. Furthermore, the arrayed B-cell epitopes can be recognized as foreign and induce B-cell activation to produce protective neutralizing antibodies against surface antigens.

Monomeric fusion proteins are disclosed herein that include the following elements linked in an N-terminal to C-terminal direction: (1) hepatitis B surface antigen; (2) a linear linking peptide; and, (3) an antigenic polypeptide including at least one epitope of the MPR, wherein the antigenic polypeptide is not full-length gp41, gp120, or gp160. Generally the monomeric fusion proteins form a self-aggregating multimeric ring structure upon expression in a host cell. Similarly, the monomeric fusion proteins can assemble spontaneously (self-aggregate) when placed in suspension in a solution of physiological pH (for example, a pH of about 7.0 to 7.6). Thus, in the present disclosure, wherever a monomeric fusion protein is disclosed, polymeric forms are also considered to be described.

The monomeric fusion proteins disclosed herein include hepatitis B surface antigen as the N-terminal member of the fusion protein. Suitable amino acid sequences for hepatitis B surface antigen are known in the art, and are disclosed, for example, in PCT Publication No. WO 2002/079217, which is incorporated herein by reference. Additional sequences for hepatitis B surface antigen can be found, for example, in PCT Publication No. 2004/113369 and PCT Publication No. WO 2004/09849. An exemplary HBsAg amino acid sequence, and the sequence of a nucleic acid encoding HBsAg, is shown in Berkower et al., Virology 321: 74-86, 2004, which is incorporated herein by reference. The sequence of a nucleic acid encoding HBsAg polypeptide is set forth in SEQ ID NO:30. The amino acid sequence of an HBsAg is set forth in SEQ ID NO:31.

By itself, HBsAg assembles into approximately 22 nm virus-like particles. When expressed together with an HIV-1 antigenic epitope, the HSBsAg fusion proteins assemble spontaneously and efficiently into virus-like particles (see Berkower et al., Virology 321: 75-86, 2004, which is incorporated herein by reference). Without being bound by theory, the multimeric form expresses the one or more antigenic epitopes at the lipid-water interface. These epitopes can be used to induce an immune response, such as to induce the production of neutralizing antibodies.

The preparation of hepatitis B surface antigen (HBsAg) is well documented. See, for example, Harford et al. (1983) Develop. Biol. Standard 54:125; Greg et al. (1987) Biotechnology 5:479; EP-A-0 226 846; and EP-A-0 299 108.

Fragments and variants of hepatitis B surface antigen are also encompassed. By “fragment” of a hepatitis B surface antigen is intended a portion of a nucleotide sequence encoding a hepatitis B surface antigen, or a portion of the amino acid sequence of the protein. By “homologue” or “variant” is intended a nucleotide or amino acid sequence sufficiently identical to the reference nucleotide or amino acid sequence, respectively. Included are those fragments and variants that retain the ability to spontaneously assemble into virus-like particles.

It is recognized that the gene or cDNA encoding a polypeptide can be considerably mutated without materially altering one or more the polypeptide's functions. The genetic code is well known to be degenerate, and thus different codons encode the same amino acids. Even where an amino acid substitution is introduced, the mutation can be conservative and have no material impact on the essential functions of a protein (see Stryer, Biochemistry 4th Ed., W. Freeman & Co., New York, N.Y., 1995). Part of a polypeptide chain can be deleted without impairing or eliminating all of its functions. Sequence variants of a protein, such as a 5′ or 3′ variant, can retain the full function of an entire protein. Moreover, insertions or additions can be made in the polypeptide chain for example, adding epitope tags, without impairing or eliminating its functions (Ausubel et al., Current Protocols in Molecular Biology, Greene Publ. Assoc. and Wiley-Intersciences, 1998). Other modifications that can be made without materially impairing one or more functions of a polypeptide include, for example, in vivo or in vitro chemical and biochemical modifications or the incorporation of unusual amino acids. Such modifications include, for example, acetylation, carboxylation, phosphorylation, glycosylation, ubiquination, labeling, such as with radionuclides, and various enzymatic modifications, as will be readily appreciated by those well skilled in the art. A variety of methods for labeling polypeptides and labels useful for such purposes is well known in the art, and includes radioactive isotopes such as ³²P, ligands that bind to or are bound by labeled specific binding partners (such as antibodies), fluorophores, chemiluminescent agents, enzymes, and antiligands.

Functional fragments and variants of hepatitis B surface antigen include those fragments and variants that are encoded by nucleotide sequences that retain the ability to spontaneously assemble into virus-like particles. Functional fragments and variants can be of varying length. For example, a fragment may consist of 10 or more, 25 or more, 50 or more, 75 or more, 100 or more, or 200 or more amino acid residues of a hepatitis B surface antigen amino acid sequence.

A functional fragment or variant of hepatitis B surface antigen is defined herein as a polypeptide that is capable of spontaneously assembling into virus-like particles and/or self-aggregating into stable multimers. This includes, for example, any polypeptide six or more amino acid residues in length that is capable of spontaneously assembling into virus-like particles. Methods to assay for virus-like particle formation are well known in the art (see, for example, Berkower et al. (2004) Virology 321:75-86, herein incorporated by reference in its entirety).

“Homologues” or “variants” of a hepatitis B surface antigen are encoded by a nucleotide sequence sufficiently identical to a nucleotide sequence of hepatitis B surface antigen, examples of which are described above. By “sufficiently identical” is intended an amino acid or nucleotide sequence that has at least about 60% or 65% sequence identity, about 70% or 75% sequence identity, about 80% or 85% sequence identity, about 90%, 91%, 92%, 93%, 94%, 95%, 96%, 97%, 98% or 99% sequence identity over its full length as compared to a reference sequence, for example using the NCBI Blast 2.0 gapped BLAST set to default parameters. Alignment may also be performed manually by inspection. For comparisons of amino acid sequences of greater than about 30 amino acids, the Blast 2 sequences function is employed using the default BLOSUM62 matrix set to default parameters (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). In one embodiment, the HBsAg protein is at least about 85%, at least about 90%, at least about 95%, at least about 96%, at least about 97%, at least about 98%, at least about 99% identical to the polypeptide encoded by the nucleotide sequence set forth as SEQ ID NO:30.

One or more conservative amino acid modifications can be made in the HBsAg amino acid sequence, whether an addition, deletion or modification, that does not substantially alter the 3-dimensional structure of the polypeptide. For example, a conservative amino acid substitution does not affect the ability of the HBsAg polypeptide to self-aggregate into stable multimers. HBsAg proteins having deletions of a small number of amino acids, for example, less than about 10% (such as less than about 8%, or less than about 5%, or less than about 2%, or less than about 1%) of the total number of amino acids in the wild type HBsAg protein can also be included in the fusion proteins described herein. The deletion may be a terminal deletion, or an internal deletion, so long as the deletion does not substantially affect the structure or aggregation of the fusion protein.

Between the self-aggregating Hepatitis B surface antigen polypeptide component and the antigenic polypeptide, the monomeric fusion protein includes a linker sequence or linear linking peptide. This peptide is a short amino acid sequence providing a flexible linker that permits attachment of an antigenic epitope without disruption of the structure, aggregation (multimerization) or activity of the self-aggregating polypeptide component. Typically, a linear linking peptide consists of between two and 25 amino acids. Usually, the linear linking peptide is between two and 15 amino acids in length. In one example, the linker polypeptide is two to three amino acids in length, such as a serine and an arginine, or two serine residues and an arginine residue, or two arginine residues and a serine residue.

In other examples, the linear linking peptide can be a short sequence of alternating glycines and prolines, such as the amino acid sequence glycine-proline-glycine-proline. A linking peptide can also consist of one or more repeats of the sequence glycine-glycine-serine. Alternatively, the linear linking peptide can be somewhat longer, such as the glycine(4)-serine spacer described by Chaudhary et al., Nature 339:394-397, 1989.

Directly or indirectly adjacent to the remaining end of the linear linking peptide (that is, the end of the linear linking peptide not attached to the self-aggregating polypeptide component of the fusion protein) is a polypeptide sequence including at least one antigenic epitope of HIV-1, such as an epitope of gp41, such as at least one antigenic epitope of the membrane proximal region. The antigenic polypeptide can be a short peptide sequence including a single epitope. For example the antigenic polypeptide can be a sequence of amino acids as short as eight or nine amino acids, sufficient in length to provide an antigenic epitope in the context of presentation by a cellular antigen presenting complex, such as the major histocompatibility complex (MHC). The antigenic polypeptide can also be of sufficient in length to induce antibodies, such as neutralizing antibodies. Larger peptides, in excess of 10 amino acids, 20 amino acids or 30 amino acids are also suitable antigenic polypeptides, as are much larger polypeptides provided that the antigenic polypeptide does not disrupt the structure or aggregation of the HBsAg polypeptide component. It should be noted that in several embodiments, the antigenic polypeptide does not include a full length gp41, gp120, or pg160 amino acid sequence. In one example, the antigenic polypeptide does not include at least 500 amino acids of gp120, such as the amino acid sequence utilized by Berkower et al., Virology 321: 75-86, 2004, incorporated herein by reference).

Exemplary embodiments ranging from short HIV-1 peptides (for example, less than 20 amino acids in length) to longer polypeptides (such as about 120, about 150 amino acids, or about 200 amino acids), including multiple antigenic epitopes are described in the examples herein. In several examples, the antigenic peptide includes one or more epitopes of the envelope protein of HIV-1, and is about 20 to about 200 amino acids in length, such as about 25 to about 150 amino acids in length, such as about 25 to about 100 amino acids in length. In several additional examples, the antigenic polypeptide includes one or more antigenic epitopes of HIV-1 gp41, such as the membrane proximal region (MPR) of gp41.

Exemplary sequences for HIV-1, as well as the amino acid sequences for full-length gp41, gp120 and gp160 can be found on Genbank, EMBL and SwissProt websites. Exemplary non-limiting sequence information can be found for example, as SwissProt Accession No. P04578, (includes gp41 and gp120, initial entry Aug. 13, 1987, last modified on Jul. 15, 1999); Genbank Accession No. HIVHXB2CG (full length HIV-1, including RNA sequence and encoded proteins, Oct. 21, 2002); Genbank Accession No. CAD23678 (gp41, Apr. 15, 2005); Genbank Accession No. AAF69493 (Oct. 2, 2000, gp120); Genbank Accession No. CAA65369 (Apr. 18, 2005); all of which are incorporated herein by reference. Similar information is available for HIV-2. Generally, the membrane proximal region of gp41 is considered to be residues 655 to 683 of gp41.

Suitable Env proteins are known in the art and include, for example, gp160, gp120, gp41, and gp140. Any clade of HIV is appropriate for antigen selection, including HIV clades A, B, C, and the like. HIV Gag, Pol, Nef and/or Env proteins from HIV clades A, B, C, as well as nucleic acid sequences encoding such proteins and methods for the manipulation and insertion of such nucleic acid sequences into vectors, are known (see, for example, HIV Sequence Compendium, Division of AIDS, National Institute of Allergy and Infectious Diseases, 2003, HIV Sequence Database (on the world wide web at hiv-web.lanl.gov/content/hiv-db/mainpage.html), Sambrook et al., Molecular Cloning, a Laboratory Manual, 2d edition, Cold Spring Harbor Press, Cold Spring Harbor, N.Y., 1989, and Ausubel et al., Current Protocols in Molecular Biology, Greene Publishing Association. Exemplary Env polypeptides, for example, corresponding to clades A, B and C are represented by the sequences of Genbank® Accession Nos. U08794, K03455 and AF286227, respectively.

In one example, the antigenic epitope comprises the amino acid sequence of NEX₁X₂LLX₃LDKWASLWNWFDITNWLWYIK (SEQ ID NO:1, consensus of MPR). In this sequence, X₁, X₂ and X₃ are any amino acid. The antigenic epitope can include repeats of this sequence, such as one to five copies of SEQ ID NO:1. As noted above, the antigenic peptide includes one or more epitopes of the envelope protein of HIV-1, and, including SEQ ID NO:1, can be from about 28 to about 200 amino acids in length, such from about 28 to about 150 amino acids in length, such as from about 28 to about 140 amino acids in length.

In several examples, the antigenic epitope includes one or more of the amino acid sequences set forth below:

| | | |
|---|---|---|
| a) | SEQ ID NO: 2 | (NEQELLALDKWASLWNWFDITNWLWYIK); |
| b) | SEQ ID NO: 3 | (NEQDLLALDKWASLWNWFDITNWLWYIK); |
| c) | SEQ ID NO: 4 | (NEQDLLALDKWANLWNWFDISNWLWYIK); |
| d) | SEQ ID NO: 5 | (NEQDLLALDKWANLWNWFNITNWLWYIR); |
| e) | SEQ ID NO: 6 | (NEQELLELDKWASLWNWFDITNWLWYIK); |
| f) | SEQ ID NO: 7 | (NEKDLLALDSWKNLWNWFDITNWLWYIK); |
| g) | SEQ ID NO: 8 | (NEQDLLALDSWENLWNWFDITNWLWYIK); |
| h) | SEQ ID NO: 9 | (NEQELLELDKWASLWNWFSITQWLWYIK); |
| i) | SEQ ID NO: 10 | (NEQELLALDKWASLWNWFDISNWLWYIK); |
| j) | SEQ ID NO: 11 | (NEQDLLALDKWDNLWSWFTITNWLWYIK); |
| k) | SEQ ID NO: 12 | (NEQDLLALDKWASLWNWFDITKWLWYIK); |
| l) | SEQ ID NO: 13 | (NEQDLLALDKWASLWNWFSITNWLWYIK); |
| m) | SEQ ID NO: 14 | (NEKDLLELDKWASLWNWFDITNWLWYIK); |
| n) | SEQ ID NO: 15 | (NEQEILALDKWASLWNWFDISKWLWYIK); |
| o) | SEQ ID NO: 16 | (NEQDLLALDKWANLWNWFNISNWLWYIK); |
| p) | SEQ ID NO: 17 | (NEQDLLALDKWASLWSWFDISNWLWYIK); |
| q) | SEQ ID NO: 18 | (NEKDLLALDSWKNLWSWFDITNWLWYIK); |
| r) | SEQ ID NO: 19 | (NEQELLQLDKWASLWNWFSITNWLWYIK); |
| s) | SEQ ID NO: 20 | (NEQDLLALDKWASLWNWFDISNWLWYIK); |
| t) | SEQ ID NO: 21 | (NEQELLALDKWASLWNWFDISNWLWYIR); |
| or | | |
| u) | SEQ ID NO: 22 | (NEQELLELDKWASLWNWFNITNWLWYIK). |

The antigenic epitope can include one of the amino acid sequences set forth as SEQ ID NOs:2-22. A single copy of one of SEQ ID NOs:2-22 can be included as the antigenic epitope. Alternatively, multiple copies of one of SEQ ID NOs:2-22 can be included as the antigenic epitope. Thus, one, two, three, four or five copies of one of the amino acid sequences set forth as SEQ ID NOs:2-22 can be included as the antigenic epitope.

In additional embodiments, more than one of these sequences can be included in the antigenic epitope. Thus, in several examples, two, three, four of five of the amino acid sequences set forth as SEQ ID NOs:2-22 can be included as the antigenic epitope. Each amino acid sequences included in the antigenic epitope can be present only a single time, or can be repeated.

In another example, the monomeric fusion protein includes the following polypeptides, linked in an N-terminal to C-terminal direction: (1) a hepatitis B surface antigen (2) a linear linking peptide; and (3) an antigenic polypeptide including one to five repeats of the amino acid sequence of the amino acid sequence of the 2F5 epitope, EQXLLXLDKWASLWGG (SEQ ID NO:23), wherein X is any amino acid. In several examples, the antigenic polypeptide does not include amino acids 1 to 500 of a gp160 amino acid sequence (SEQ ID NO:25). In one specific, non-limiting example, X is glutamine.

The monomeric fusion protein can optionally include hydrophobic amino acids C-terminal to the antigenic polypeptide. For example, the monomeric fusion protein can include about five to about twenty-five hydrophobic amino acids, such as about five, about ten, about fifteen, about twenty or about twenty five hydrophobic amino acid residues. Exemplary amino acids sequences include IFIMI (SEQ ID NO:26), IFIMIVGGLV (SEQ ID NO:27), IFIMIVGGLVGLRLV (SEQ ID NO:28), IFIMIVGGLVGLRLVFSIETGG (SEQ ID NO:29). The monomeric fusion protein can optionally include basic amino acids C-terminal to the antigenic polypeptide. For example, the monomeric fusion protein can include about five to about twenty-five basic amino acids, such as about five, about ten, about fifteen, about twenty, or about 25 basic amino acid residues. Examples of groups of basic amino acids that can be used include, but are not limited to, HRKKR (SEQ ID NO:57) and HRKRHKRRKH (SEQ ID NO:58). The monomeric fusion protein can also optionally include a suitable T cell epitope. Generally, a T cell epitope is about eight to about ten amino acids in length, such as about nine amino acid in length, and binds major histocompatibility complex (MHC), such as HLA 2, for example, HLA 2.2. Examples of suitable T cell epitopes include, but are not limited to, ASLWNWFNITNWLWY (SEQ ID NO:32) and IKLFIMIVGGLVGLR (SEQ ID NO:33).

The monomeric fusion protein may also include a CAAX (SEQ ID NO:34) sequence, for isoprenyl addition in vivo. In this sequence, C is cysteine, A is an aliphatic amino acid and X is any amino acid. The X residue determines which isoprenoid will be added to the cysteine. When X is a methionine or serine, the farnesyl-transferase transfers a farnesyl, and when X is a leucine or isoleucine, the geranygeranyl-transferase I, a geranylgeranyl group. In general, aliphatic amino acids have protein side chains containing only carbon or hydrogen atoms. Aliphatic amino acids include proline (P), glycine (G), alanine (A), valine (V), leucine (L), and isoleucine (I), presented in order from less hydrophobic to more hydrophobic. Although methionine has a sulphur atom in its side-chain, it is largely non-reactive, meaning that methionine effectively substitutes well with the true aliphatic amino acids.

### Polynucleotides Encoding Monomeric Fusion Polypeptides

Nucleic acids encoding the monmeric fusion proteins described herein are also provided. These nucleic acids include deoxyribonucleotides (DNA, cDNA) or ribodeoxynucleotides (RNA) sequences, or modified forms of either nucleotide, which encode the fusion polypeptides described herein. The term includes single and double stranded forms of DNA and/or RNA. The nucleic acids can be operably linked to expression control sequences, such as, but not limited to, a promoter.

The nucleic acids that encode the monomeric fusion protein disclosed herein include a polynucleotide sequence that encodes a monomeric fusion protein including a hepatitis B surface antigen polypeptide, a linker and an antigenic epitope of the envelope protein of HIV, such as an epitope of gp41, gp120 or gp160, wherein the nucleic acid does not encode full length gp41, gp120 or gp160. The fusion proteins and the polynucleotides encoding them described herein can be used to produce pharmaceutical compositions, including compositions suitable for prophylactic and/or therapeutic administration. These compositions can be used to induce an immune response to HIV, such as a protective immune response. However, the compositions can also be used in various assays, such as in assays designed to detect an HIV-1 infection.

Methods and plasmid vectors for producing the polynucleotides encoding fusion proteins and for expressing these polynucleotides in bacterial and eukaryotic cells are well known in the art, and specific methods are described in Sambrook et al. (In Molecular Cloning: A Laboratory Manual, Ch. 17, CSHL, New York, 1989). Such fusion proteins may be made in large amounts, are easy to purify, and can be used to elicit an immune response, including an antibody response and/or a T cell response. Native proteins can be produced in bacteria by placing a strong, regulated promoter and an efficient ribosome-binding site upstream of the cloned gene. If low levels of protein are produced, additional steps may be taken to increase protein production; if high levels of protein are produced, purification is relatively easy. Suitable methods are presented in Sambrook et al (In Molecular Cloning: A Laboratory Manual, CSHL, New York, 1989) and are well known in the art. Often, proteins expressed at high levels are found in insoluble inclusion bodies. Methods for extracting proteins from these aggregates are described by Sambrook et al. (In Molecular Cloning: A Laboratory Manual, Ch. 17, CSHL, New York, 1989). Proteins, including fusion proteins, may be isolated from protein gels, lyophilized, ground into a powder and used as an antigen.

Vector systems suitable for the expression of polynucleotides encoding fusion proteins include, in addition to the specific vectors described in the examples, the pUR series of vectors (Ruther and Muller-Hill, EMBO J. 2:1791, 1983), pEX1-3 (Stanley and Luzio, EMBO J. 3:1429, 1984) and pMR100 (Gray et al., Proc. Natl. Acad. Sci. USA 79:6598, 1982). Vectors suitable for the production of intact native proteins include pKC30 (Shimatake and Rosenberg, Nature 292:128, 1981), pKK177-3 (Amann and Brosius, Gene 40:183, 1985) and pET-3 (Studiar and Moffatt, J. Mol. Biol. 189:113, 1986), as well as the pCMV/R vector disclosed in the Examples section below. The CMV/R promoter is described in, among other places, PCT Application No. PCT/US02/30251 and PCT Publication No. WO03/028632.

The DNA sequence can also be transferred from its existing context to other cloning vehicles, such as other plasmids, bacteriophages, cosmids, animal viruses and yeast artificial chromosomes (YACs) (Burke et al., Science 236:806-812, 1987). These vectors may then be introduced into a variety of hosts including somatic cells, and simple or complex organisms, such as bacteria, fungi (Timberlake and Marshall, Science 244:1313-1317, 1989), invertebrates, plants (Gasser and Fraley, Science 244:1293, 1989), and animals (Pursel et al., Science 244:1281-1288, 1989), which cell or organisms are rendered transgenic by the introduction of the heterologous cDNA. Specific, non-limiting examples of host cells include mammalian cells (such as CHO or HEK293 cells), insect cells (Hi5 or SF9 cells) or yeast cells.

For expression in mammalian cells, a cDNA sequence may be ligated to heterologous promoters, such as the simian virus (SV) 40 promoter in the pSV2 vector (Mulligan and Berg, Proc. Natl. Acad. Sci. USA 78:2072-2076, 1981), or the cytomegalovirus promoter, and introduced into cells, such as monkey COS-1 cells (Gluzman, Cell 23:175-182, 1981), to achieve transient or long-term expression. The stable integration of the chimeric gene construct may be maintained in mammalian cells by biochemical selection, such as neomycin (Southern and Berg, J. Mol. Appl. Genet. 1:327-341, 1982) and mycophenolic acid (Mulligan and Berg, Proc. Natl. Acad. Sci. USA 78:2072-2076, 1981).

DNA sequences can be manipulated with standard procedures such as restriction enzyme digestion, fill-in with DNA polymerase, deletion by exonuclease, extension by terminal deoxynucleotide transferase, ligation of synthetic or cloned DNA sequences, site-directed sequence-alteration via single-stranded bacteriophage intermediate or with the use of specific oligonucleotides in combination with PCR or other in vitro amplification.

A cDNA sequence (or portions derived from it) such as a cDNA encoding a monomeric fusion protein can be introduced into eukaryotic expression vectors by conventional techniques. These vectors are designed to permit the transcription of the cDNA in eukaryotic cells by providing regulatory sequences that initiate and enhance the transcription of the cDNA and ensure its proper splicing and polyadenylation. Vectors containing the promoter and enhancer regions of the SV40 or long terminal repeat (LTR) of the Rous Sarcoma virus and polyadenylation and splicing signal from SV40 are readily available (Mulligan et al., Proc. Natl. Acad. Sci. USA 78:1078-2076, 1981; Gorman et al., Proc. Natl. Acad. Sci. USA 78:6777-6781, 1982). The level of expression of the cDNA can be manipulated with this type of vector, either by using promoters that have different activities (for example, the baculovirus pAC373 can express cDNAs at high levels in S. frugiperda cells (Summers and Smith, In Genetically Altered Viruses and the Environment, Fields et al. (Eds.) 22:319-328, CSHL Press, Cold Spring Harbor, N.Y., 1985) or by using vectors that contain promoters amenable to modulation, for example, the glucocorticoid-responsive promoter from the mouse mammary tumor virus (Lee et al., Nature 294:228, 1982). The expression of the cDNA can be monitored in the recipient cells 24 to 72 hours after introduction (transient expression).

In addition, some vectors contain selectable markers such as the gpt (Mulligan and Berg, Proc. Natl. Acad. Sci. USA 78:2072-2076, 1981) or neo (Southern and Berg, J. Mol. Appl. Genet. 1:327-341, 1982) bacterial genes. These selectable markers permit selection of transfected cells that exhibit stable, long-term expression of the vectors (and therefore the cDNA). The vectors can be maintained in the cells as episomal, freely replicating entities by using regulatory elements of viruses such as papilloma (Sarver et al., Mol. Cell. Biol. 1:486, 1981) or Epstein-Barr (Sugden et al., Mol. Cell. Biol. 5:410, 1985). Alternatively, one can also produce cell lines that have integrated the vector into genomic DNA. Both of these types of cell lines produce the gene product on a continuous basis. One can also produce cell lines that have amplified the number of copies of the vector (and therefore of the cDNA as well) to create cell lines that can produce high levels of the gene product (Alt et al., J. Biol. Chem. 253:1357, 1978).

The transfer of DNA into eukaryotic, in particular human or other mammalian cells, is conventional. The vectors are introduced into the recipient cells as pure DNA (transfection) by, for example, precipitation with calcium phosphate (Graham and vander Eb, Virology 52:466, 1973) or strontium phosphate (Brash et al., Mol. Cell. Biol. 7:2013, 1987), electroporation (Neumann et al., EMBO J. 1:841, 1982), lipofection (Felgner et al., Proc. Natl. Acad. Sci. USA 84:7413, 1987), DEAE dextran (McCuthan et al., J. Natl. Cancer Inst. 41:351, 1968), microinjection (Mueller et al., Cell 15:579, 1978), protoplast fusion (Schafner, Proc. Natl. Acad. Sci. USA 77:2163-2167, 1980), or pellet guns (Klein et al., Nature 327:70, 1987). Alternatively, the cDNA, or fragments thereof, can be introduced by infection with virus vectors. Systems are developed that use, for example, retroviruses (Bernstein et al., Gen. Engr'g 7:235, 1985), adenoviruses (Ahmad et al., J. Virol. 57:267, 1986), or Herpes virus (Spaete et al., Cell 30:295, 1982). Polynucleotides that encode proteins, such as fusion proteins, can also be delivered to target cells in vitro via non-infectious systems, for instance liposomes.

Using the above techniques, the expression vectors containing a polynucleotide encoding a monomeric fusion protein as described herein or cDNA, or fragments or variants or mutants thereof, can be introduced into human cells, mammalian cells from other species or non-mammalian cells as desired. The choice of cell is determined by the purpose of the treatment. For example, monkey COS cells (Gluzman, Cell 23:175-182, 1981) that produce high levels of the SV40 T antigen and permit the replication of vectors containing the SV40 origin of replication may be used. Similarly, Chinese hamster ovary (CHO), mouse NIH 3T3 fibroblasts or human fibroblasts can be used.

The present disclosure, thus, encompasses recombinant vectors that comprise all or part of the polynucleotides encoding self-aggregating monomeric fusion proteins or cDNA sequences, for expression in a suitable host, either alone or as a labeled or otherwise detectable protein. The DNA is operatively linked in the vector to an expression control sequence in the recombinant DNA molecule so that the fusion polypeptide or protein can be expressed. The expression control sequence may be selected from the group consisting of sequences that control the expression of genes of prokaryotic or eukaryotic cells and their viruses and combinations thereof. The expression control sequence may be specifically selected from the group consisting of the lac system, the trp system, the tac system, the trc system, major operator and promoter regions of phage lambda, the control region of fd coat protein, the early and late promoters of SV40, promoters derived from polyoma, adenovirus, retrovirus, baculovirus and simian virus, the promoter for 3-phosphoglycerate kinase, the promoters of yeast acid phosphatase, the promoter of the yeast alpha-mating factors and combinations thereof.

Any host cell can be transfected with the vector of this disclosure. Exemplary host cells include, but are not limited to E. coli, Pseudomonas, Bacillus subtilis, Bacillus stearothermophilus or other bacilli; other bacteria; yeast; fungi; insect; mouse or other animal; plant hosts; or human tissue cells.

Multimeric forms of a monomeric fusion protein ring can be recovered (such as for administration to a subject, or for other purposes) using any of a variety of methods known in the art for the purification of recombinant polypeptides. The monomeric fusion proteins disclosed herein can produced efficiently by transfected cells and can be recovered in quantity using any purification process known to those of skill in the art, such as a nickel (NTA-agarose) affinity chromatography purification procedure.

A variety of common methods of protein purification may be used to purify the disclosed fusion proteins. Such methods include, for instance, protein chromatographic methods including ion exchange, gel filtration, HPLC, monoclonal antibody affinity chromatography and isolation of insoluble protein inclusion bodies after over production. As described in further detail in the examples, in a favorable embodiment one or more purification affinity-tags, for instance a six-histidine sequence, is recombinantly fused to the protein and used to facilitate polypeptide purification (optionally, in addition to another functionalizing portion of the fusion, such as a targeting domain or another tag, or a fluorescent protein, peptide, or other marker).

Commercially produced protein expression/purification kits provide tailored protocols for the purification of proteins made using each system. See, for instance, the QIAEXPRESS™ expression system from QIAGEN (Chatsworth, Calif.) and various expression systems provided by INVITROGEN (Carlsbad, Calif.). Where a commercial kit is employed to produce an APOBEC3G fusion protein, the manufacturer's purification protocol is a preferred protocol for purification of that protein. For instance, proteins expressed with an amino-terminal hexa-histidine tag can be purified by binding to nickel-nitrilotriacetic acid (Ni-NTA) metal affinity chromatography matrix (The QIAexpressionist, QIAGEN, 1997).

### Therapeutic Methods and Pharmaceutical Compositions

Polynucleotides encoding the monomeric fusion proteins disclosed herein, and monomeric fusion proteins, and the stable multimeric ring structures formed by polypeptides expressed from such polynucleotides can be administered to a subject in order to generate an immune response to HIV-1. In one example, the immune response is a protective immune response. Thus, the polynucleotides and polypeptides disclosed herein can be used in a vaccine, such as a vaccine to prevent subsequent infection with HIV.

A therapeutically effective amount of monomeric fusion protein, a polymeric form thereof, a viral particle including these fusion proteins, or a polynucleotide encoding one or more of these polypeptides can be administered to a subject to prevent, inhibit or to treat a condition, symptom or disease, such as acquired immunodeficiency syndrome (AIDS). In one example, polymeric ring structures formed by monomeric fusion protein subunits are administered. In another example, one or more polynucleotides encoding at least one fusion polypeptide are administered. As such, the fusion polypeptides and polynucleotides encoding fusion polypeptides can be administered as vaccines to prophylactically or therapeutically induce or enhance an immune response. For example, the pharmaceutical compositions described herein can be administered to stimulate a protective immune response against HIV, such as a HIV-1.

A single administration can be utilized to prevent or treat an HIV infection, or multiple sequential administrations can be performed. In another example, more than one of the monomeric fusion polypeptides, multimeric forms of more than one monomeric fusion polypeptides, or multiple polynucleotides encoding the monomeric fusion polypeptides, including different antigenic epitopes as described above, are administered to a subject to induce an immune response to HIV-1. These polypeptides or polynucleotides can be administered simultaneously, or sequentially.

In exemplary applications, compositions are administered to a subject infected with HIV, or likely to be exposed to an infection, in an amount sufficient to raise an immune response to HIV. Administration induces a sufficient immune response to reduce viral load, to prevent or lessen a later infection with the virus, or to reduce a sign or a symptom of HIV infection. Amounts effective for this use will depend upon various clinical parameters, including the general state of the subject's health, and the robustness of the subject's immune system, amongst other factors. A therapeutically effective amount of the compound is that which provides either subjective relief of one or more symptom(s) of HIV infection, an objectively identifiable improvement as noted by the clinician or other qualified observer, a decrease in viral load, an increase in lymphocyte count, such as an increase in CD4 cells, or inhibit development of symptoms associated with infection.

The monomeric fusion protein, multimeric forms of the monomeric fusion proteins and polynucleotides encoding them can be administered by any means known to one of skill in the art (see Banga, A., “Parenteral Controlled Delivery of Therapeutic Peptides and Proteins,” in Therapeutic Peptides and Proteins, Technomic Publishing Co., Inc., Lancaster, Pa., 1995) such as by intramuscular, subcutaneous, or intravenous injection, but even oral, nasal, or anal administration is contemplated. Monomeric fusion proteins, polymeric forms thereof, viral particles including the fusion proteins, or polynucleotides encoding the monomeric fusion proteins can be administered in a formulation including a carrier or excipient. A wide variety of suitable excipients are known in the art, including physiological phosphate buffered saline (PBS), and the like. Optionally, the formulation can include additional components, such as aluminum hydroxylphophosulfate, alum, diphtheria CRM₁₉₇, or liposomes. To extend the time during which the peptide or protein is available to stimulate a response, the peptide or protein can be provided as an implant, an oily injection, or as a particulate system. The particulate system can be a microparticle, a microcapsule, a microsphere, a nanocapsule, or similar particle. A particulate carrier based on a synthetic polymer has been shown to act as an adjuvant to enhance the immune response, in addition to providing a controlled release. Aluminum salts may also be used as adjuvants to produce an immune response.

In one embodiment, the monomeric fusion protein or multimeric form thereof is mixed with an adjuvant containing two or more of a stabilizing detergent, a micelle-forming agent, and an oil. Suitable stabilizing detergents, micelle-forming agents, and oils are detailed in U.S. Pat. No. 5,585,103; U.S. Pat. No. 5,709,860; U.S. Pat. No. 5,270,202; and U.S. Pat. No. 5,695,770, all of which are incorporated by reference. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, ZWITTERGENT™ 3-12, TEEPOL HB7™, and SPAN 85™. These detergents are usually provided in an amount of approximately 0.05 to 0.5%, such as at about 0.2%. A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by BASF Wyandotte publications, for example, Schmolka, J. Am. Oil. Chem. Soc. 54:110, 1977; and Hunter et al., J. Immuol 129:1244, 1981, PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett, J. Immun. 133:3167, 1984. The agent can be provided in an effective amount, for example between 0.5 and 10%, or in an amount between 1.25 and 5%.

The oil included in the composition is chosen to promote the retention of the antigen in oil-in-water emulsion, such as to provide a vehicle for the desired antigen, and preferably has a melting temperature of less than 65° C. such that emulsion is formed either at room temperature (about 20° C. to 25° C.), or once the temperature of the emulsion is brought down to room temperature. Examples of such oils include squalene, Squalane, EICOSANE™, tetratetracontane, glycerol, and peanut oil or other vegetable oils. In one specific, non-limiting example, the oil is provided in an amount between 1 and 10%, or between 2.5 and 5%. The oil should be both biodegradable and biocompatible so that the body can break down the oil over time, and so that no adverse affects, such as granulomas, are evident upon use of the oil.

An adjuvant can be included in the composition. In one example, the adjuvant is a water-in-oil emulsion in which antigen solution is emulsified in mineral oil (such as Freund's incomplete adjuvant or montanide-ISA). In one embodiment, the adjuvant is a mixture of stabilizing detergents, micelle-forming agent, and oil available under the name PROVAX® (IDEC Pharmaceuticals, San Diego, Calif.).

In another embodiment, a pharmaceutical composition includes a nucleic acid encoding one or more monomeric fusion protein(s) as disclosed herein. A therapeutically effective amount of the immunogenic polynucleotide can be administered to a subject in order to generate an immune response, such as a protective immune response.

One approach to administration of nucleic acids is direct immunization with plasmid DNA, such as with a mammalian expression plasmid. As described above, the nucleotide sequence encoding an NSP2-fusion protein can be placed under the control of a promoter to increase expression of the molecule. Suitable vectors are described, for example, in U.S. Pat. No. 6,562,376.

Immunization by nucleic acid constructs is well known in the art and taught, for example, in U.S. Pat. No. 5,643,578 (which describes methods of immunizing vertebrates by introducing DNA encoding a desired antigen to elicit a cell-mediated or a humoral response), and U.S. Pat. No. 5,593,972 and U.S. Pat. No. 5,817,637 (which describe operatively linking a nucleic acid sequence encoding an antigen to regulatory sequences enabling expression). U.S. Pat. No. 5,880,103 describes several methods of delivery of nucleic acids encoding immunogenic peptides or other antigens to an organism. The methods include liposomal delivery of the nucleic acids, and immune-stimulating constructs, or ISCOMS™, negatively charged cage-like structures of 30-40 nm in size formed spontaneously on mixing cholesterol and QUIL A™ (saponin). Protective immunity has been generated in a variety of experimental models of infection, including toxoplasmosis and Epstein-Barr virus-induced tumors, using ISCOMS™ as the delivery vehicle for antigens (Mowat and Donachie, Immunol. Today 12:383, 1991). Doses of antigen as low as 1 μg encapsulated in ISCOMS™ have been found to produce Class I mediated CTL responses (Takahashi et al., Nature 344:873, 1990).

In another approach to using nucleic acids for immunization, a monomeric fusion protein as disclosed herein can also be expressed by an attenuated viral host or vector, or a bacterial vector. Recombinant adeno-associated virus (AAV), herpes virus, retrovirus, or other viral vectors can be used to express the peptide or protein, thereby eliciting a CTL response.

In one embodiment, a nucleic acid encoding the monomeric fusion protein is introduced directly into cells. For example, the nucleic acid may be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites, including tissues subject to or in proximity to a site of infection. Dosages for injection are usually around 0.5 μg/kg to about 50 mg/kg, and typically are about 0.005 mg/kg to about 5 mg/kg (see, e.g., U.S. Pat. No. 5,589,466).

In one specific, non-limiting example, a pharmaceutical composition for intravenous administration, would include about 0.1 μg to 10 mg of a monomeric fusion protein per subject per day. Dosages from 0.1 pg to about 100 mg per subject per day can be used, particularly if the agent is administered to a secluded site and not into the circulatory or lymph system, such as into a body cavity or into a lumen of an organ. Actual methods for preparing administrable compositions will be known or apparent to those skilled in the art and are described in more detail in such publications as Remingtons Pharmaceuticals Sciences, 19^{th} Ed., Mack Publishing Company, Easton, Pa. (1995).

The compositions can be administered, either systemically or locally, for therapeutic treatments, such as to treat an HIV infection. In therapeutic applications, a therapeutically effective amount of the composition is administered to a subject infected with HIV, such as, but not limited to, a subject exhibiting signs or symptoms of AIDS. Single or multiple administrations of the compositions can be administered depending on the dosage and frequency as required and tolerated by the subject. In one embodiment, the dosage is administered once as a bolus, but in another embodiment can be applied periodically until a therapeutic result is achieved. Generally, the dose is sufficient to treat or ameliorate symptoms or signs of the HIV infection without producing unacceptable toxicity to the subject.

Controlled release parenteral formulations can be made as implants, oily injections, or as particulate systems. For a broad overview of protein delivery systems, see Banga, Therapeutic Peptides and Proteins: Formulation, Processing, and Delivery Systems, Technomic Publishing Company, Inc., Lancaster, Pa. (1995). Particulate systems include microspheres, microparticles, microcapsules, nanocapsules, nanospheres, and nanoparticles. Microcapsules contain the therapeutic protein as a central core. In microspheres, the therapeutic agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly (see Kreuter, Colloidal Drug Delivery Systems, J. Kreuter, ed., Marcel Dekker, Inc., New York, N.Y., pp. 219-342 (1994); Tice & Tabibi, Treatise on Controlled Drug Delivery, A. Kydonieus, ed., Marcel Dekker, Inc. New York, N.Y., pp. 315-339 (1992)).

Polymers can be used for ion-controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature. It has shown to be an effective vehicle for formulation and sustained delivery of recombinant interleukin-2 and urease (Johnston et al., Pharm. Res. 9:425, 1992; and Pec, J. Parent. Sci. Tech. 44(2):58, 1990). Alternatively, hydroxyapatite has been used as a microcarrier for controlled release of proteins (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic proteins are known (e.g., U.S. Pat. No. 5,055,303; U.S. Pat. No. 5,188,837; U.S. Pat. No. 4,235,871; U.S. Pat. No. 4,501,728; U.S. Pat. No. 4,837,028; U.S. Pat. No. 4,957,735; and U.S. Pat. No. 5,019,369; U.S. Pat. No. 5,055,303; U.S. Pat. No. 5,514,670; U.S. Pat. No. 5,413,797; U.S. Pat. No. 5,268,164; U.S. Pat. No. 5,004,697; U.S. Pat. No. 4,902,505; U.S. Pat. No. 5,506,206; U.S. Pat. No. 5,271,961; U.S. Pat. No. 5,254,342; and U.S. Pat. No. 5,534,496).

### Immunodiagnostic Reagents and Kits

In addition to the therapeutic methods provided above, any of the monomeric fusion proteins disclosed herein can be utilized to produce antigen specific immunodiagnostic reagents, for example, for serosurveillance. Without being bound by theory, antigenic peptides presented in the context of a monomeric fusion polypeptide possess a greater freedom of movement, and therefore, greater accessibility to antibody and ligands that peptides directly bound to a substrate (for example, as in common ELISA procedures). This provides increased sensitivity without a loss of specificity when the fusion polypeptide is employed in an immunoassay, such as a radioimmunoassay (“RIA”) or an enzyme-based immunoassay (“EIA”).

Immunodiagnostic reagents can be designed from any of the antigenic polypeptide described herein. For example, the presence of serum antibodies to HIV can be monitored using the monomeric fusion polypeptides disclosed herein. Thus, the monomeric fusion proteins disclosed herein, and polymeric forms thereof, can be used to detect an HIV infection. Generally, the method includes contacting a sample from a subject, such as, but not limited to a blood, serum, plasma, urine or sputum sample from the subject with one or more of the monomeric fusion proteins disclosed herein (or a polymeric form thereof) and detecting binding of antibodies in the sample to the monomeric fusion protein (or the polymeric form thereof). The binding can be detected by any means known to one of skill in the art, including the use of labeled secondary antibodies that specifically bind the antibodies from the sample. Labels include radiolabels, enzymatic labels, and fluorescent labels.

Any such immunodiagnostic reagents can be provided as components of a kit. Optionally, such a kit includes additional components including packaging, instructions and various other reagents, such as buffers, substrates, antibodies or ligands, such as control antibodies or ligands, and detection reagents.

The disclosure is illustrated by the following non-limiting Examples.

### EXAMPLES

### Example 1

### Biochemical Analysis of Recombinant HBsAg-MPR and Variants

Materials and Methods

Construction of HbsAg-MPR Variants

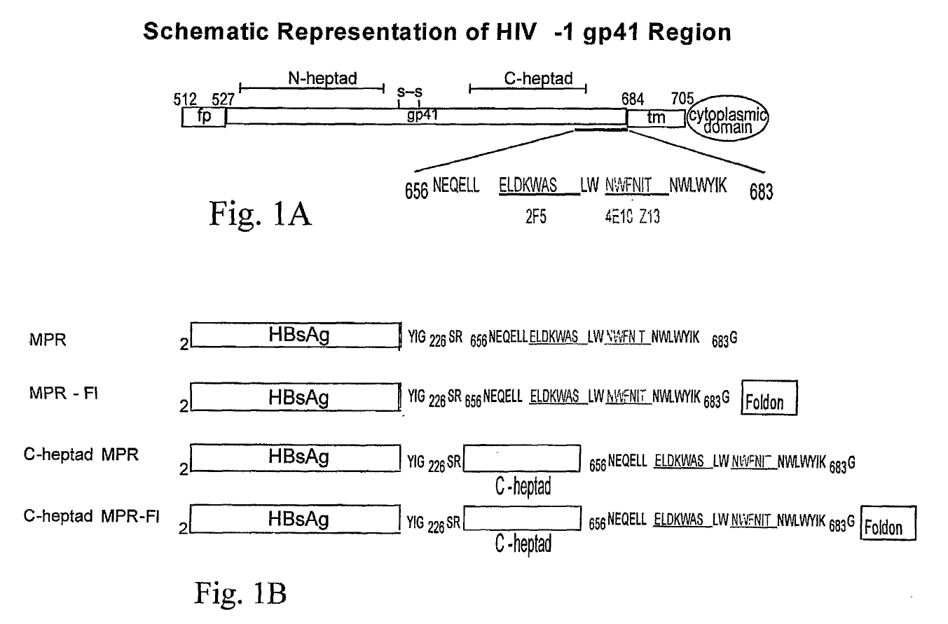

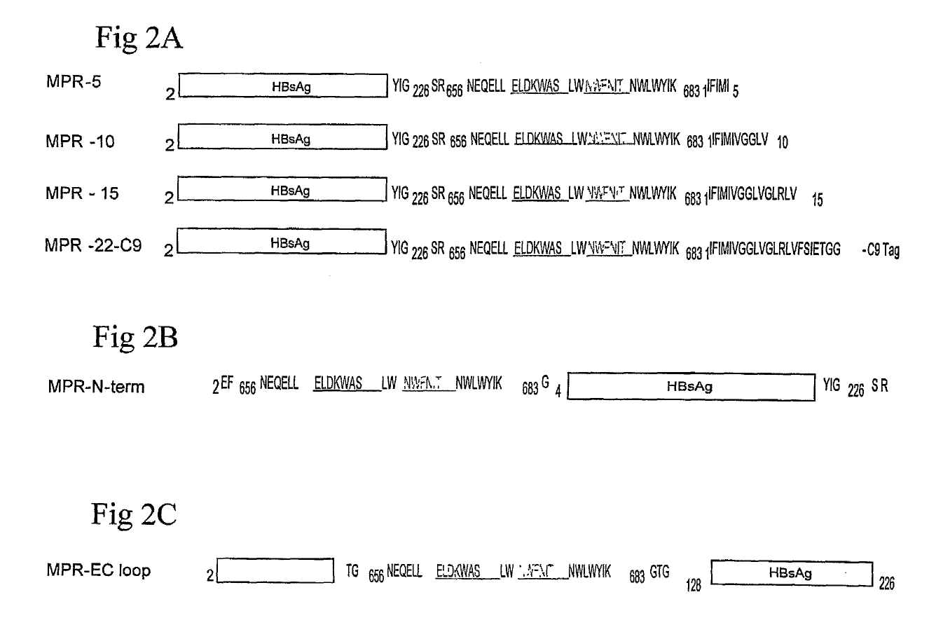

Amino acids 2 to 226 of the synthetic S gene HBsAg (Berkower et al. (2004) Virology 321:75-86) were used as a scaffold to implant the membrane proximal regions of HIV-1 gp41 at the N-terminus, C-terminus or the extra-cellular loop of the HBsAg. The S gene of HBsAg was amplified from the vector pGEM using primers forward primer 5′ GGA GCTCGT CGA CAG CAA 3′ (SEQ ID NO:38) and reverse primer 5′GCT CTA GAC CCG ATG TAG ACC CA 3′ (SEQ ID NO:39) to introduce a SalI site at the 5′ end and an XbaI site at the 3′ end of the gene. The amplified product was cloned into a pCMV/R vector at the SalI and XbaI sites. Variants of gp41 sequences were amplified using codon-optimized HIV-1 Yu2 gp160 or JRFLg160 as the template. (See Table 1 for the list of primers used). The initial set of constructs was generated with HIV-1 gp41 region, C-heptad and/or the membrane proximal region at the C-terminus of HBsAg. Between the HBsAg and the gp41 region two amino acids (S and R) were introduced, and at the end of Lysine 683 a glycine was placed immediately before the stop codon. The T4 fibritin trimerization domain, foldon was also introduced in two of the constructs to determine the effect of trimerization on recombinant HBsAg particle production and recognition of 2F5 and 4E10 (see FIG. 1B). The second set of constructs was generated to introduce various lengths of HIV-1 transmembrane region after the lysine 683 of the MPR, ₁IFIMI₅ (SEQ ID NO:26) for MPR-5, ₁IFIMIVGGLV₁₀ (SEQ ID NO:27) for MPR-10, ₁IFIMIVGGLVGLRLV₁₅ (SEQ ID NO:28) for MPR-15 and ₁IFIMIVGGLVGLRLVFSIETGG₂₂ TETSQVAPA (SEQ ID NO:29)-C9 tag for MPR-22-C9 in order to further stabilize and orient the 4E10 epitope (see FIG. 2A). The third set of constructs was generated by placing the MPR at the N-terminus after the 2^{nd} and 3^{rd} amino acid (EF) of the HBsAg sequence. A further modification of this set of constructs was to clone a transmembrane sequence to the N-terminus of the MPR in order to restrict the free movement of MPR and to provide a lipid membrane context for 2F5 epitope (see FIG. 2B). A final set of constructs was generated by creating an AgeI site by replacing P₁₂₆ and A₁₂₇ with TG. The MPR with a 3 amino acid linker (GTG) at the C-terminus of MPR was cloned at the AgeI site to place it in the extra cellular loop (EC loop) of HBsAg. The EC loop is the most immunogenic and neutralization determinant of HBsAg (see FIG. 2C).

Cell Line and Transfection

One day prior to transfection, 8 million HEK 293T cells in DMEM, 10% FBS, 1% penicillin-streptomycin (pen-strep) were seeded in a 150 mm tissue culture dish. The cells were transfected with the plasmids encoding recombinant HBsAg-MPR and MPR variants, and wild type HBsAg, using Fugene6 (Roche) at a ratio DNA:Fugene6 1:3 and 10 μg/plate.

Particle Production and Analysis

The constructs were transfected into HEK293T cells. Four to five days after transfection, cells and supernatant were collected. Supernatant was concentrated using Centricon Plus-80 100 kDa Biomax memb (Millipore, Billerica, Mass.) to 25 mls. The cells were lysed by resuspending them in 10 ml of 1×PBS and sonicating for 1 min at 20 Hz every 10 sec using a probe sonicator. Following this the cell lysate was cleared at 15000 rpm for 15 mins. The concentrated supernatant and the cell lysate were loaded on a 20% sucrose cushion (20% sucrose in PBS) and centrifuged at 23000 rpm for 16 hrs (Surespin rotor, Sorvall). The partially purified VLPs were resuspended in PBS and analyzed by ELISA or Western blotting. To further purify them, the resuspended VLPs were loaded onto a 10-40% (wt/wt) CsCl stp gradient (in PBS) and centrifuged at 22 h at 36000 rpm (TV-860 rotor, Sorvall), and 500□μl fractions were taken from the bottom of the tube. The fractions containing VLPs were identified by ELISA. The positive fractions were desalted, concentrated, and washed with PBS using Amicon YM-100 filter (Millipore).

ELISA Assay for HBsAg

To detect the presence of HBsAg, HBsAg-MPR and MPR variant VLPs in the preparations, ELISA was performed. For direct ELISA the particles were adsorbed onto a high-protein-binding microwell plate (Corning) for 2 hrs, then blocked with the blocking buffer (PBS with 2% dry milk). After one wash with PBS/0.2% Tween-20, anti-HBsAg antibody NE3 or NF5 (Aldevron) was added to each well as a serial dilution and incubated at 37° C. for 1 hr. After three washes with PBS/0.2% Tween-20, a secondary Anti-Mouse-IgG-HRP antibody (Sigma) was added in washing buffer at a 1:5000 dilution for 1 h at 37° C. Following three washes, the ELISAs were developed with 100 μl TMB Peroxidase substrate (KPL). The reaction was stopped by adding 100 μl 11 M HCl to each well. The optical density at 450-nm was read on a microplate reader (Molecular Devices).

For sandwich ELISA, 500 ng of mouse monoclonal NE3 antibody (Aldevron) was adsorbed onto each well overnight at 4° C. and then blocked with the blocking buffer. Then the particles were resuspended in PBS and 100 μl of each was added to each well and incubated at 37° C. for 2 hrs. After one wash with PBS/0.2% Tween-20, antibody 2F5, 4E10 (kindly provided by H Katinger), HIVIgG (NIH AIDS Reagent Repository Program) or HIV-1 positive human sera was added to each well as a serial dilution and incubated at 37° C. for 1 hr. After three washes with PBS/0.2% Tween-20, a secondary anti-human-IgG-HRP antibody (Jackson Immuno Research labs) was added in washing buffer at a 1:5000 dilution for 1 h at 37° C. Following three washes, the ELISAs were developed as described above.

For competition ELISA, all the steps similar to those for the sandwich ELISA were performed except that the peptide NEQELLELDKWASLWN (SEQ ID NO:40) was mixed along with 2F5 or serially diluted human sera and incubated at 37° C. for 1 hr.

Results

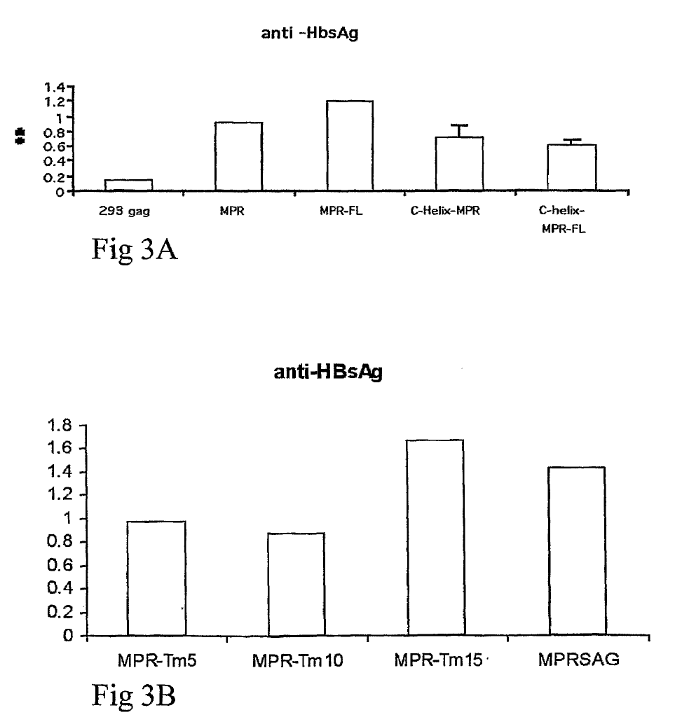

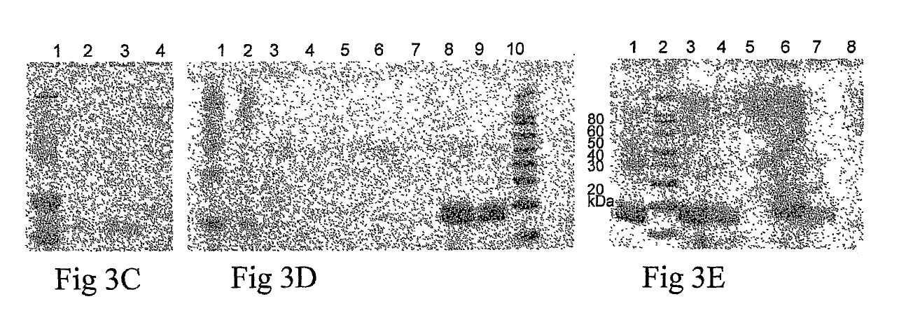

The individual plasmids containing the HBsAg-MPR variant constructs were transfected into HEK293T cells. 293 gag particles were used as negative control. Five days after transfection, tissue culture supernatants and cell pellets were collected for isolation of particles. Recombinant particles were pelleted by centrifugation through a 20% sucrose cushion and were purified further by CsCl gradient. The particles were tested either by direct or sandwich ELISA utilizing an HbsAg-specific capture antibody, NE3. All the constructs expressed and generated recombinant particles except construct MPR-22-C9 (FIGS. 3A and B). Particle production was markedly reduced for the constructs with longer exogenous sequences, such as C-heptad-MPR-containing constructs. This has been observed previously for recombinant HBsAg-gp120 particles, suggesting that longer exogenous sequences may negatively effect particle production.

The HBsAg-MPR particles were scaled-up by the transfection of greater cell numbers, pelleted on 20% sucrose cushion and purified on CsCl gradients as described above. Each fraction was tested by ELISA and those positive for HBsAg were pooled and concentrated, and analyzed under reducing conditions on SDS gels. The yeast purified standard HBsAg monomer ran at 24 kDa. A faint dimer band and high order oligomers were also noted (see FIG. 3C). The HBsAg-MPR particles isolated by this procedure were not fully pure, but did show a predominant band that ran at 27 kDa (as expected) and a slight faint band above it which most likely is the glycosylated form (lane 4, FIG. 3C). In addition, a dimer of the S-MPR protein monomer and its glycosylated form were also observed. From the reducing Western blot analysis it was evident that the 27 kDa band, and its glycosylated forms, observable on Coomassie-blue stained SDS gels, were indeed the correct size bands as they specifically reacted to the anti-HBsAg mice polyclonal sera when tested on the recombinant HBsAg-MPR particles made from the transfected supernatant (lanes 8 and 9 FIG. 3D; lane 1 FIG. 3E) or the cell lysate (lane 3 FIG. 3E). The yeast standard HBsAg ran as a monomer 24 kDa, a dimer and high order oligomer (lanes 1 and 2 FIG. 3D and lane 8 FIG. 3D). The recombinant particle production from the supernatants of HBsAg-MPR-5 (˜27 kDa; lane 6 FIG. 3D) and MPR-15 (˜29 kDa; lane 4 FIG. 3D) were less efficient than those from the cell lysate. The recombinant particle production from HBsAg-MPR-5 was found at ˜27 kDa and a faint band of its glycosylated form was observed (lane 4 FIG. 3E). For HBsAg-MPR-15 a ˜29 kDa band was noted (lane 6 FIG. 3E). The recombinant HBsAg-MPR-10 particle production was not observed either in the cell lysate or the supernatant. It could be detected by ELISA, but particle production was reduced. The MPR-HBsAg recombinant particle also showed a ˜27 kDa monomer and its glycosylated form (lane 7 FIG. 3E).

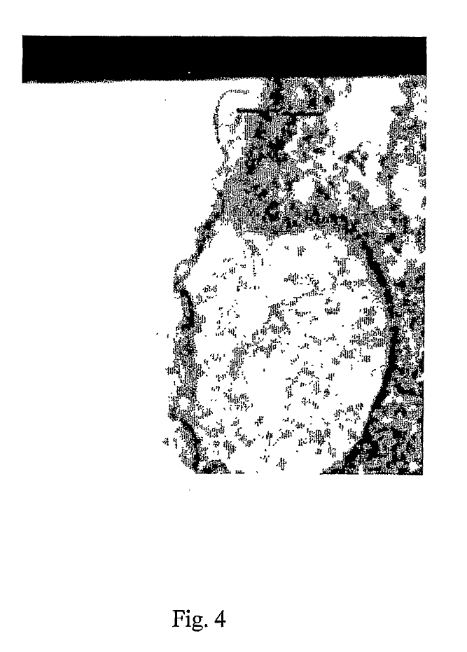

In addition, HEK295T cells were processed for electron micrography. It was determined that the HBsAg-C-term-MPR particle accumulated in the rough endoplamic reticulum in HEK293 cells (see FIG. 4).

### Example 2

### Binding of 2F5 and 4E10 to Recombinant HBsAg-MPR and Variants

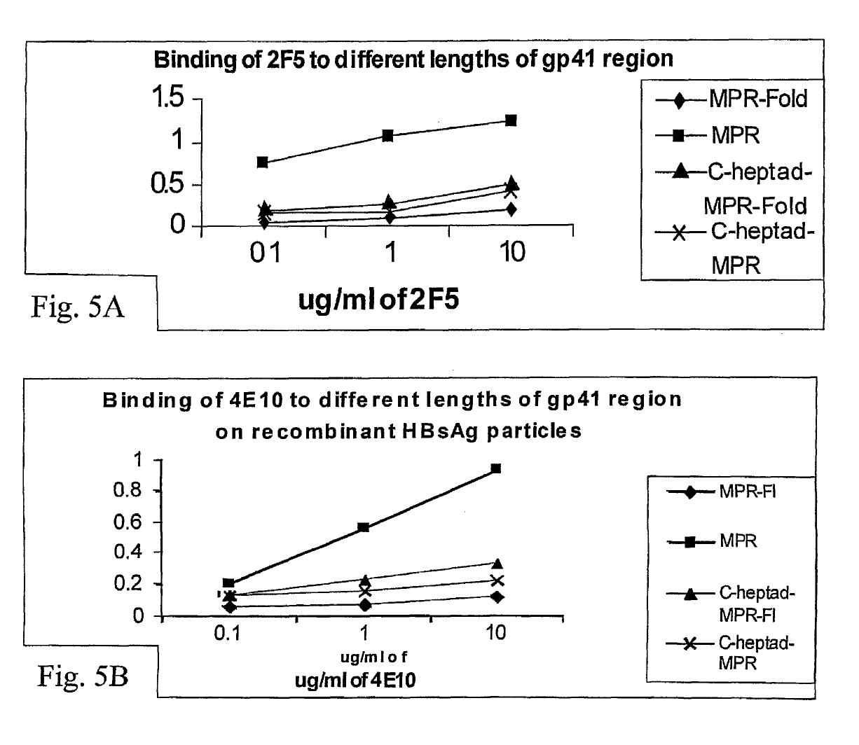

The MPR and its variants harbor the complete epitopes for both 2F5 and 4E10. The binding of 2F5 and 4E10 to the recombinant HBsAg-MPR and its variants was tested using a sandwich ELISA. From the first set of constructs (FIG. 1), HBsAg-MPR particle bound well to 2F5 and 4E10 (see FIG. 5). However, the other constructs were not well recognized by 2F5 and 4E10. The HBsAg-MPR-F1 construct did not bind to 2F5 and 4E10 suggesting that the foldon trimerization domain affected 2F5 antibody binding, perhaps by either stearic interference or by altering the 2F5 epitope. The introduction of the gp41 C-heptad repeat region upstream of the MPR affected the 2F5 and 4E10 binding either through dissociation of the epitopes from the lipid membrane or because the presence of C-heptad did not allow the C-terminus of the recombinant HBsAg to be presented at the surface.

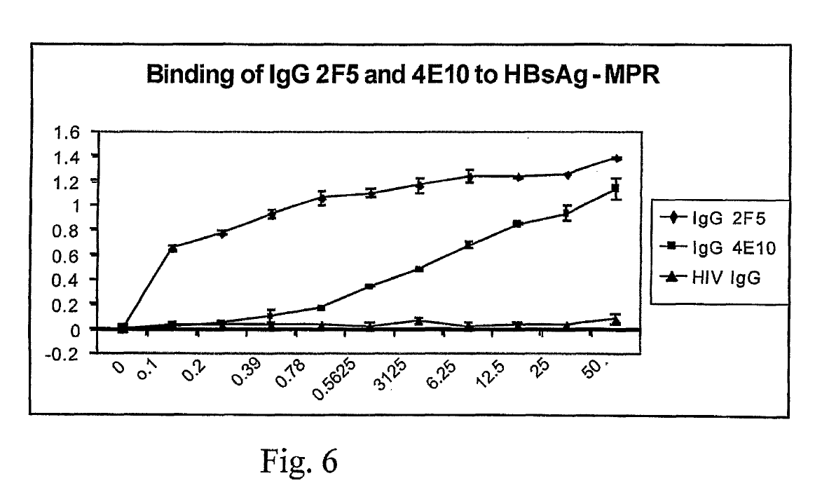

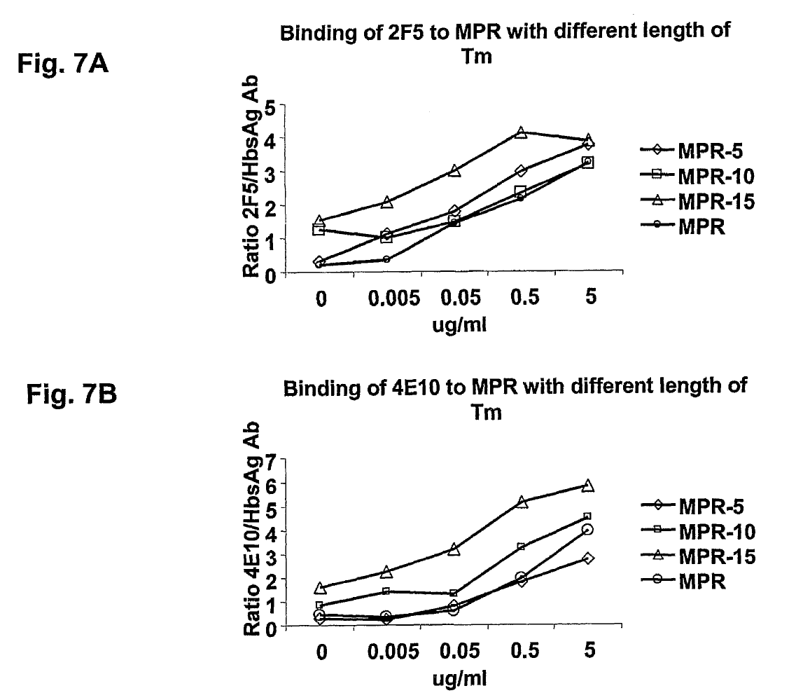

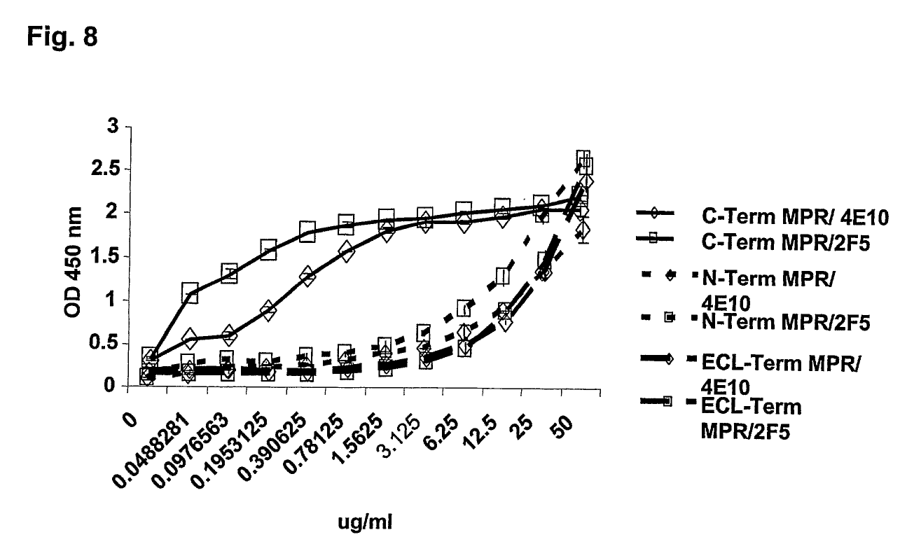

The 2F5 antibody bound with a relatively high affinity to recombinant HBsAg-MPR particles but the binding of 4E10 to these particles was relatively low (see FIG. 6). The low binding could be due to the effects on 4E10 epitope, which normally lies in a hydrophobic environment. The epitope may become hidden when it lies on the lipid membrane in the recombinant HBsAg particles. To improve 4E10 binding, different lengths of transmembrane regions following the 4E10 epitope in the MPR (5, 10, 15 and 22-C9) particles were produced. HBsAg-MPR-22-C9 particle could not be detected either by ELISA or Western blot. HBsAg-MPR-15 particles showed good relative binding to both 2F5 and 4E10 antibodies, followed by MPR-5 particles and then MPR-10 particles (see FIG. 7). The relative binding of 2F5 was best with the HBsAg-MPR particles. Binding of both 2F5 and 4E10 was good with HbsAg-MPR-15 particles (FIG. 7). In each of the above-described constructs, the MPR was placed at the C-terminus. Constructs were also generated with MPR placed at the N-terminus and at the immunodominant extracellular loop of hepatitis B surface antigen. Particles with MPR at the N-terminus and particles with MPR at the extracellular loop did not bind well to 2F5 or 4E10, as compared to particles with the MPR placed at the C-terminus (see FIG. 8). In the constructs with MPR at the N-terminus, as well as the construct with MPR at the loop, the MPR is away from the membrane (by approximately 20 to 30 amino acids), whereas in the construct with MPR at the C-terminus, the MPR is immediately in juxtaposition to the membrane. Thus, 2F5 and 4F10 may be presented in the context of membrane.

### Example 3

### Competition of 2F5 Binding to HBsAg-MPR Particles by a 16-Mer Peptide

Materials and Methods

Viral Entry Assay

Viruses YU2.5G3 and SF162.LS were mixed with peptide (85 ug/ml), HBsAg particles (0.9 mg/ml) or HBsAg-MPR particles (0.5 ug/ml) and incubated at 37° C. for 30 mins. Then 1×10⁴ TZM-B1 cells (NIH AIDS Research & Reference Reagent Program) were added per well and incubated at 37° C. overnight. The next day, the cells were lysed and luciferase expression was monitored (Luciferase Assay System, Promega) using a luminometer (Victor light luminometer; Perkin Elmer).

Neutralization Adsorption Assay

To adsorb out 2F5 neutralization activity, a 16-mer 2F5 peptide, HbsAg-MPR particles and HbsAg blank particles were used. Viruses YU2.5G3 and SF162.LS were diluted and mixed with the appropriate dilution of Ab 2F5 mixed with either peptide of HBsAg particle (serially diluted) and incubated at 37° C. for 30 mins. They were then mixed with 1×10⁴ TZM-B1 cells (NIH AIDS Research & Reference Reagent Program) per well and incubated at 37° C. overnight. The next day the cells were lysed and luciferase expression was monitored (Luciferase Assay System, Promega using a luminometer (Victor light luminometer, Perkin Elmer).

Results

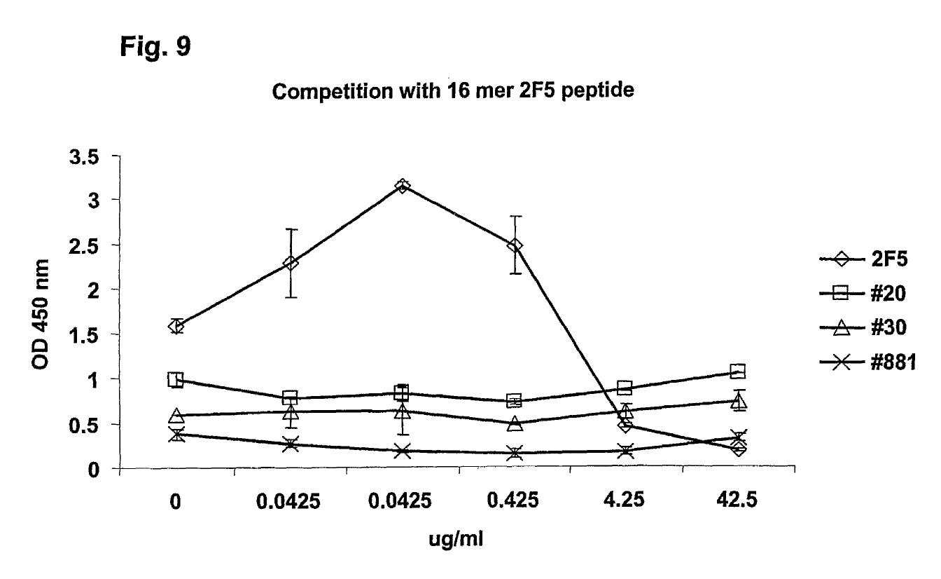

2F5 bound with a relative high affinity to HBsAg-MPR particles. To demonstrate this binding was specific a 16-mer peptide harboring the 2F5 epitope but not the 4E10 epitope was used for competition analysis. Interestingly, at low concentration of the peptide (0.00425 and 0.0425 ug/ml), the binding of 2F5 to HBsAg-MPR particle was enhanced almost 2-fold at 0.0425 ug/ml peptide (see FIG. 9). A similar effect was seen in viral entry assays where the HBsAg-MPR particle led to 2 to 2.5 fold enhanced entry YU2 and SF162 viruses. Neither the free peptide nor HBsAg particle showed this effect, suggesting a role for MPR in viral entry. At higher concentrations the peptide fully competed out 2F5 binding (see FIG. 9).

We further evaluated whether HBsAg-MPR particles that present 2F5 epitope well had the ability to adsorb out 2F5 neutralization activity. Although the particles themselves moderately enhanced viral entry, if the enhancement was taken into account, they could adsorb out ˜10% and ˜21% neutralization activity of 2F5 for YU2.5G3 virus and SF162.LS viruses respectively.

### Example 4

### Binding of HIVIgG and Human Sera from HIV-1 Positive Patients to HBsAg-MPR Particles

Materials and Methods

Human sera from HIV-1 positive patients #1, 5, 20 and 30 and antibody 2F5 were serially diluted and analyzed for binding to HBsAg and HbsAg-MPR particles in ELISA format.

Results

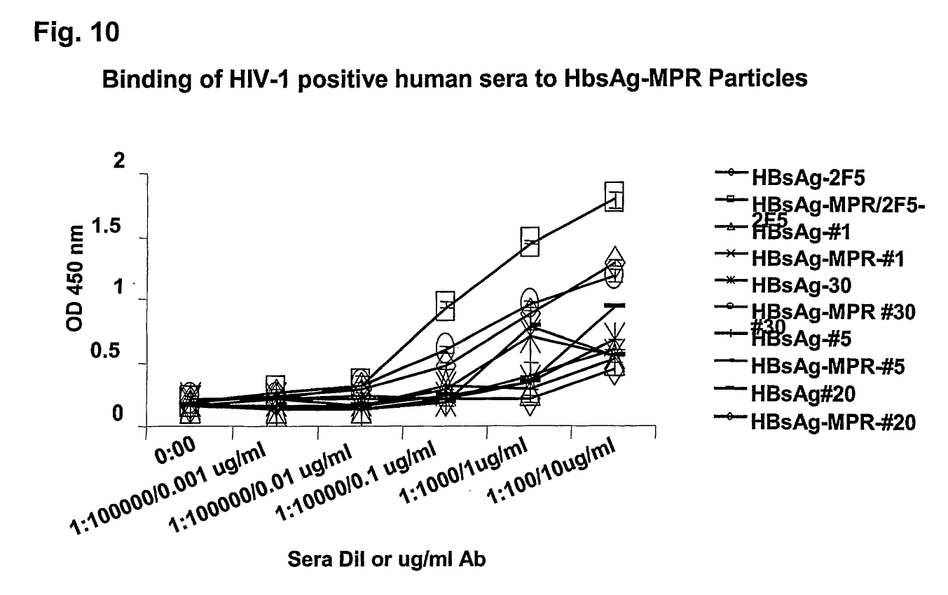

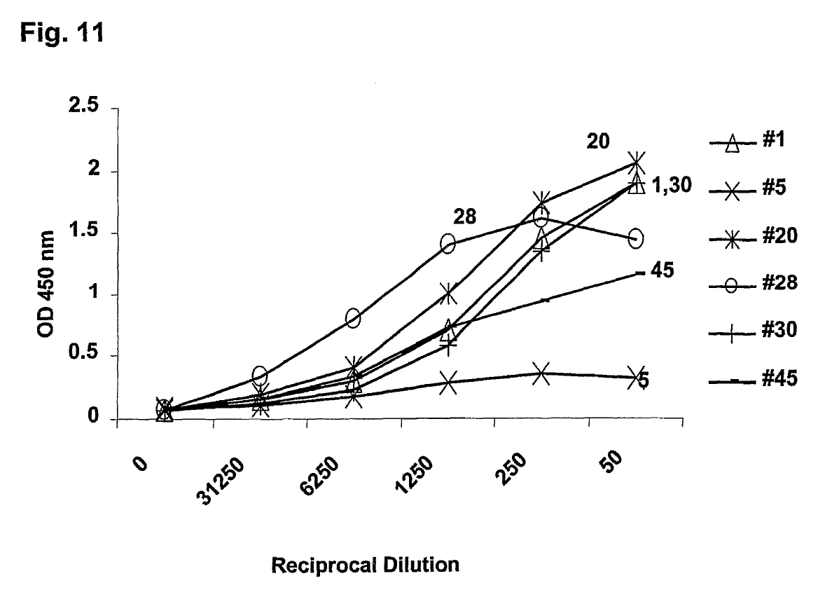

To determine the utility of HBsAg-MPR particles to identify sera that contains broad neutralizing antibodies against the MPR regions, we screened a set of weakly and broadly neutralizing human HIV-1 positive sera and HIV-IgG for binding to HBsAg-MPR particles (see FIG. 10 and Table 2). HIV-Ig, a broad-neutralizing serum tested and certified negative for HBsAg, showed no binding to HBsAg and HBsAg-MPR particles. Human sera #1, which showed broad neutralizing activity, and human sera #4 and #5 which were weak neutralizers, also did not show binding to the MPR particles. Human sera from patient #6 and #7 were moderate neutralizers and showed binding to both HBsAg and HbsAG-MPR particles. Sera #5 had slightly better binding activity to MPR particles, but it was not clear whether it had any MPR-directed activity. Human sera #20, #30 and #45 had broad-neutralization activity and also bound well to HBsAg-MPR particles, suggesting that these sera might have MPR-directed activity which could be a factor in their broad neutralization activity. Human sera #20 and #30 and 2F5 (used as a positive control) bound well to the MPR particle but not to the blank HBsAg particle. Human sera #1 and #5 showed no background binding to HBsAg and HBsAg-MPR particles. Thus, a subset of the sera that showed broad neutralization also harbored MPR-specific reactivity.

### Example 5

### MPR ELISA to Validate the HBsAG-MPR Particle Analysis of Human Sera

Materials and Methods: An ELISA assay was used to validate that antibodies in human sera ind the HBsAg-MPR. Human sera #1, #5, #20, #28, #30 and #45 were serially diluted and analyzed for binding to MPR. Anti-human secondary antibody was used for detection of signal.

Results: To further validate the utility of HBsAg-MPR particles to identify sera that contains broad neutralizing antibodies against the MPR regions, a set of weakly and broadly neutralizing human HIV-1 positive sera were screened for binding to MPR particles (see FIG. 10) in a novel MPR ELISA format. Human sera #5, which was weak neutralizer did not show binding to MPR. Whereas, human sera #1, #20, #28, #30 and #45 showed significant binding to MPR. The two analyses showed that there was notable MPR-directed activity in the sera which could be a factor in their broad neutralization activity.

### Example 6

### Effect of Lipid on the Binding of 2F5 and 4E10 to the HBsAg-C-Term-MPR Particles

Materials and Methods

HBsAG-C-term MPR particles were treated with high and low PH buffer containing detergent. Synthetic lipid (DOPC:DOPS 7:3) was exchanged into a fraction of the delipidated particles. The wild type particles, delipidated particles, and the particles with synthetic lipid were analyzed by ELISA for binding to antibodies 2F5 and 4E10 (diluted serially 0 to 10 μg/ml).

Results

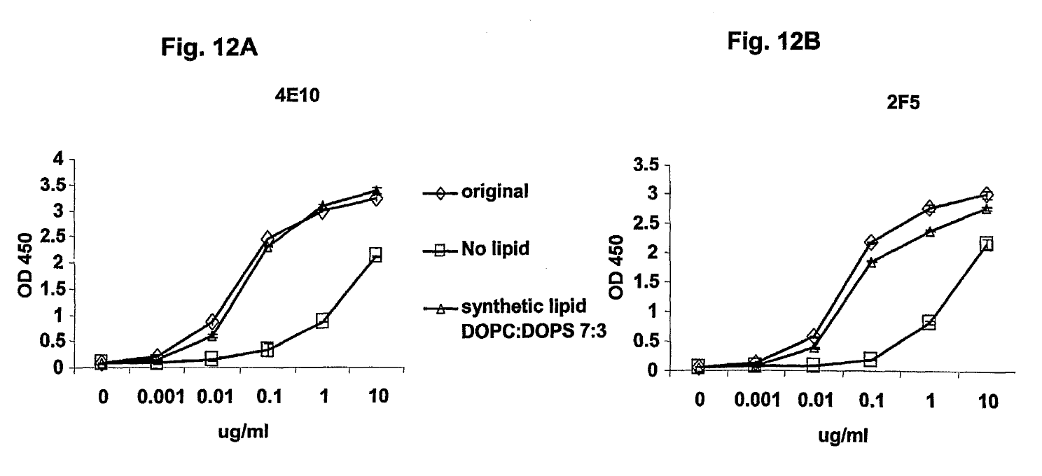

The antibodies 2F5 and 4E10 bound with relatively high affinity to wild type HBsAG-C-term-MPR particles, but when the particles were delipidated, the binding of both these antibodies was significantly reduced (see FIG. 12). On restoring the lipid component with synthetic lipids, the binding of both the antibodies was restored. Thus, the lipid context may provide the better presentation of 2F5 and 4E10 epitopes for optimal binding.

### Example 7

### Analysis of Rabbit Antisera to 2F5 Epitope-KLH

Materials and Methods

The 2F5 epitope (EQELLELDKWASLWGG) (SEQ ID NO:24) was conjugated to keyhole limpet hemocyanin (KLH) and immunized in rabbits. The sera were checked for binding to the 2F5 epitope containing peptide (1 ug/ml) coated in an ELISA plate, followed by binding of rabbit sera and 2F5 (used as positive control). The sera were also tested for cell surface binding of the sera to ADA envelope. In addition, the sera were checked for their neutralizing ability in a viral neutralization assay using sensitive HIV-1 strains and chimeric HIV-2 strains containing HIV-1 2F5 epitope.

Results

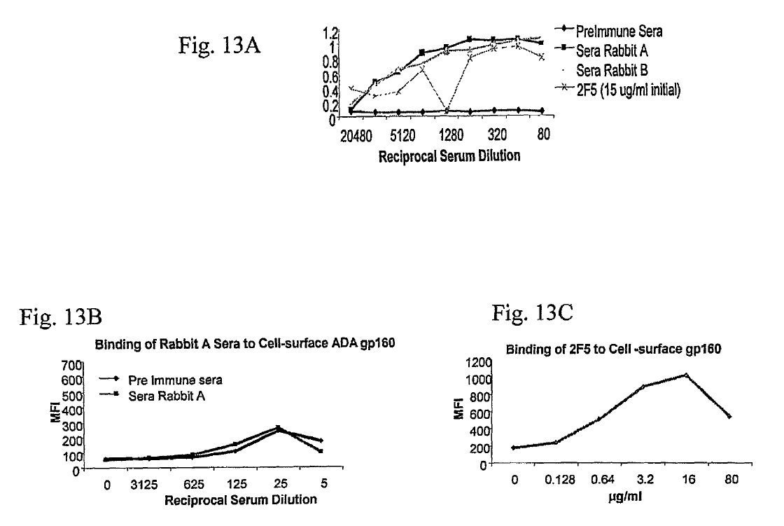

Immunized rabbits produced antibodies that recognized the 2F5 peptide (see FIG. 13A). However, this sera had no specific recognition of HIV-1 gp160 trimers expressed on the cell surface (see FIG. 13B). 2F5 was capable of binding to cell surface gp160 (see FIG. 13C). Furthermore, the rabbit sera did not have any neutralizing antibodies to sensitive HIV-1 or chimeric HIV-2 viruses, indicating that free 2F5 epitope containing peptide does not present the 2F5 epitope in the relevant context to the immune system, but raises irrelevant non-neutralizing antibodies. This analysis further emphasized the need for a relevant presentation of the 2F5 and 4E10 epitope that would allow generation of cross-reactive antibodies that could bind to envelope gp160 and neutralizing the virus.

### Example 8

### Analysis of Guinea Pig Antisera to HepB MPR Particles

Materials and Methods

Guinea pigs were immunized with 5, 20, 50 and 100 μg of the HBsAG-MPR particle in ALUM and CpG as adjuvant by i.m. route. The guinea pig sera was analyzed for binding to hepatitis B surface antigen and 2F5 epitope-containing peptide by ELISA. The sera was also analyzed for binding to the MPR-Tm in an MPR ELISA and for binding to gp160 ADA expressed on cell surface by FACS.

Results

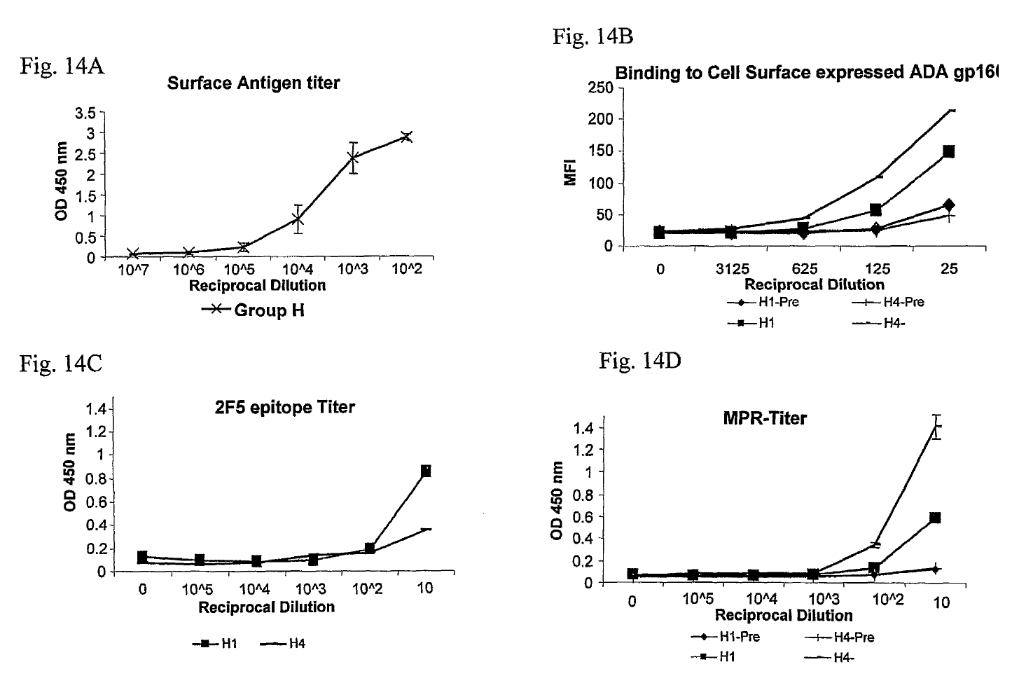

In guinea pigs immunized to HBsAg-C-term-MPR particles, a high titer of antibodies were raised to hepatitis B surface antigen. There was no significant improvement in titer beyond a dosage of 20 μg particles. Interestingly there were two groups of animals, one that showed lower titer antibodies to the 2F5 epitope-containing peptide, but bound well to MPR or gp160 ADA, and a second group that had high titer antibodies to the 2F5 epitope-containing peptide but low titer for MPR or gp160 ADA. This reciprocal effect further suggested that the MPR particles presented MPR in a relevant conformation to the immune system, thus raising antibodies which were cross-reactive in nature (see FIG. 14). The sera raised in guinea pig showed weak neutralization of sensitive HIV-1 strain SF162 but did not neutralize any other strain of HIV.

### Example 9

### Cell-Surface Binding of Antisera Elicited by HepB MPR Particles in Mice

Materials and Methods

BalB/C mice were immunized with 5 μg of the HBsAG (Control) and HBsAG-MPR particle in alum as an adjuvant by i.m. route. The mice sera were analyzed for binding to the MPR-Tm and HIV gp160 (JR-FL and YU2) expressed on cell surface. One million cells were labeled with different dilution of preimmune or the sera raised against HBsAg or HBsAg-MPR particles. After three washes with FACS buffer, the cells were labeled with anti mouse-PE labeled antibody and analyzed by FACS.

Results

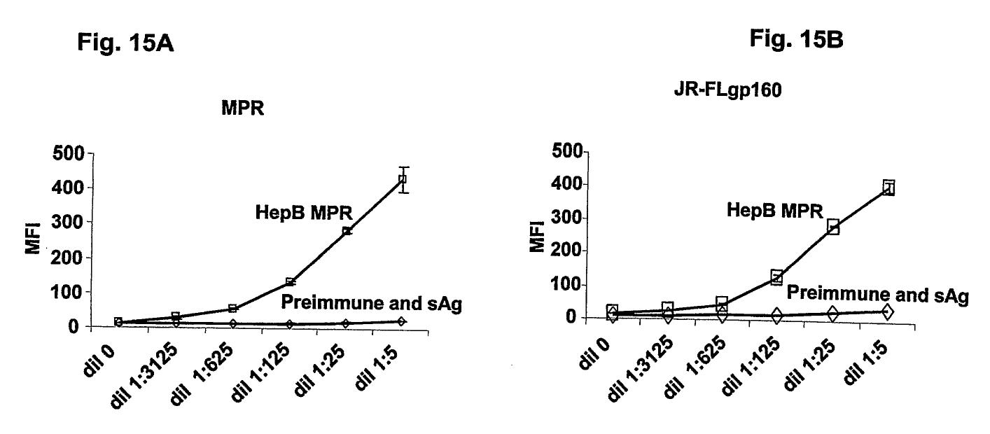

The mice sera generated against HBsAG and HBsAG-MPR particles produced good titer antibodies against the hepatitis B surface antigen but only the sera generated against HBsAG-MPR particles bound well to MPR, JR-FL and YU2 gp160 envelope glycoproteins expressed on the cell-surface (See FIG. 15). The preimmune and the HBsAG sera did not bind to MPR or the gp160 JR-FL and YU2 envelope. The cross-reactive binding of HBsAG-MPR sera to gp160 expressed on the cell surface suggested that the quality of the antibodies that the MPR particles raise was due to some relevant conformation of the 2F5 and 4E10 epitope that was presented by the HBsAG-MPR particle. Generation of cross-reactive antibodies to HIV-1 envelope gp160 by the HBsAG-MPR particle provides an avenue to utilize the immunofocusing strategy of priming with gp160 and boosting with the MPR particles or priming with the MPR particles followed by boosting with gp160, in order to specifically generate memory B cells that raise antibodies directed to the MPR region.

### Example 10

### Selection of K562 Cells Displaying Specific Abs by sAg and sAg-MPR Particles

Materials and Methods

The K562 cells contains FC receptor which were used to bind either the NF5 (mouse monoclonal antibody specific to the HBsAG) or 2F5 and 4E10 (human monoclonal antibodies) or HIVIgG (polyclonal pool of IgG from HIV positive Ig pool). The antibodies were bound to the cells at 10 μg/ml concentration. The HBsAG or the HBsAG-MPR particles were labeled with nile red (a lipid specific dye that fluoresces only after it partitions in lipids). The excess dye was dialyzed out. The cells labeled with antibodies were mixed with different concentration of the nile red-labeled HBsAG or HBsAG-MPR particles. Following three washes the cells were analyzed by FACS. Only the cells that bound to labeled particle would fluoresce, and could be sorted out.

Results

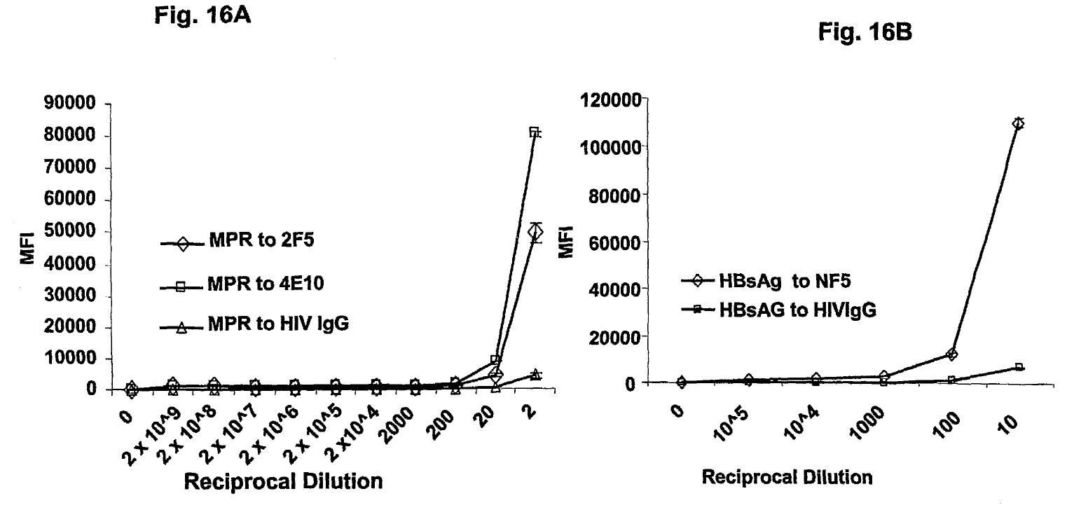

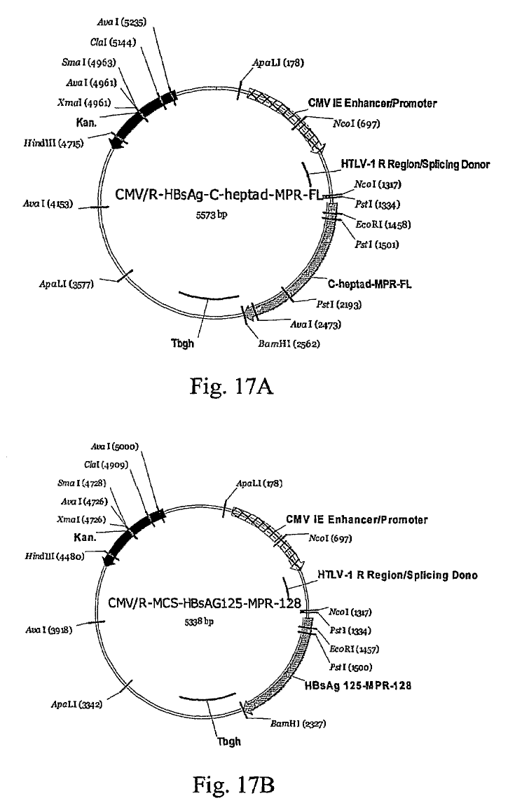

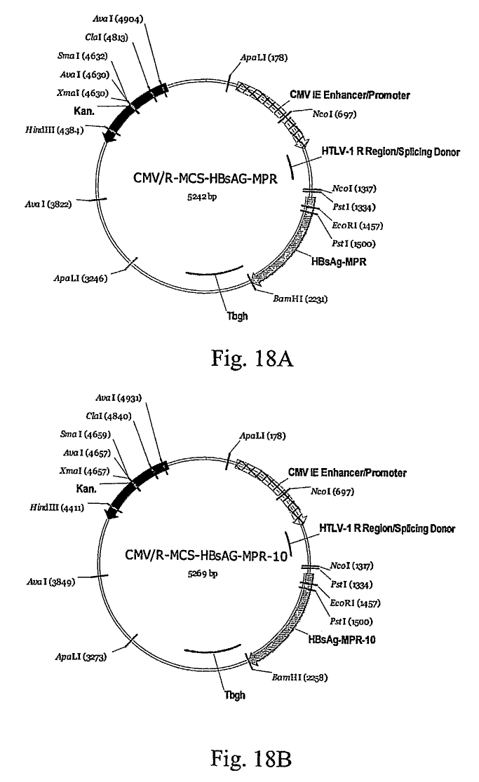

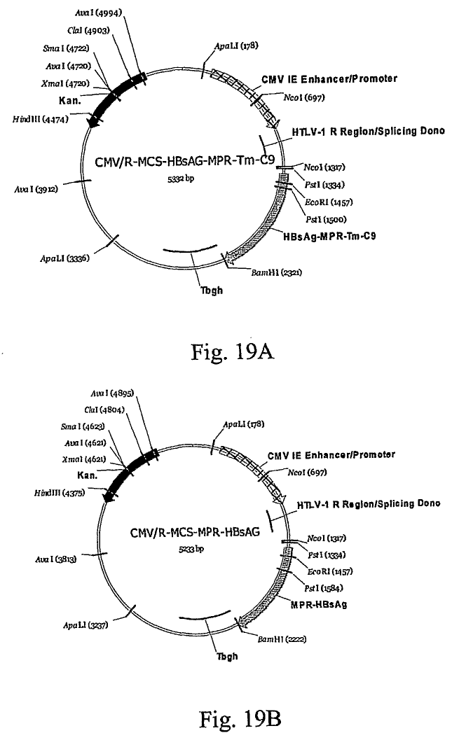

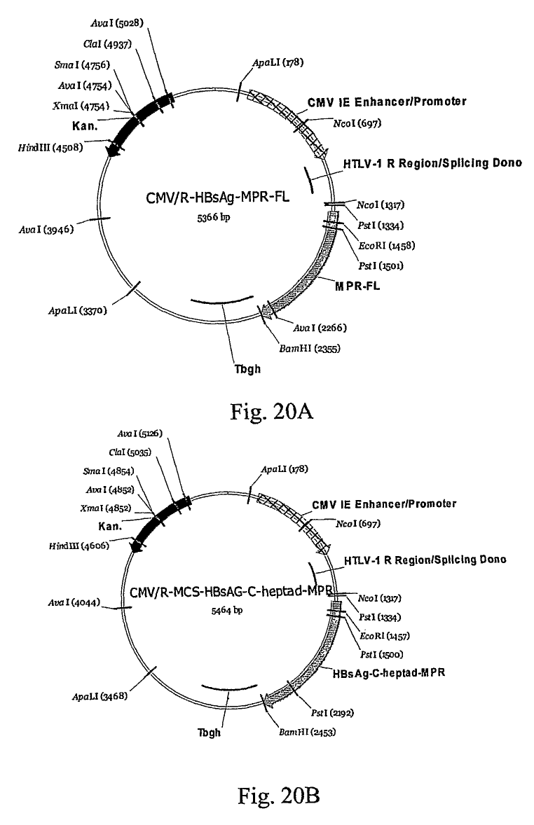

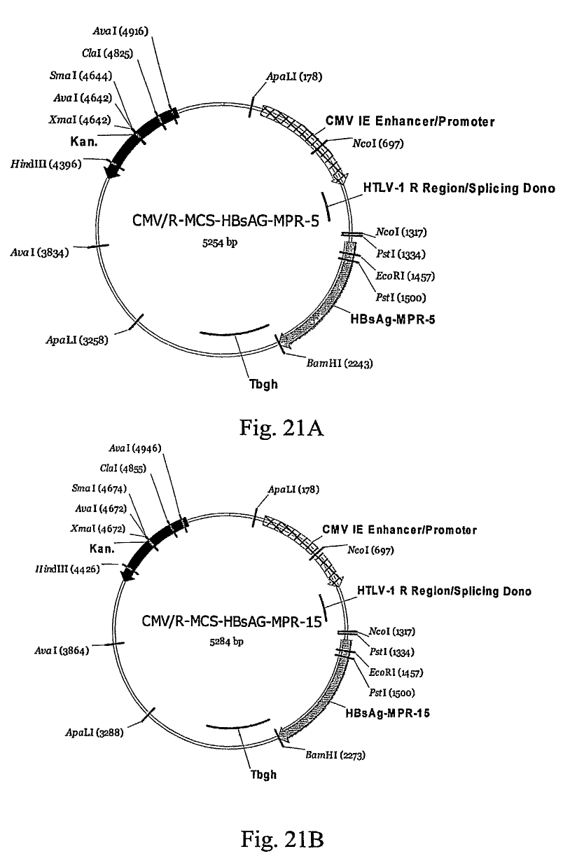

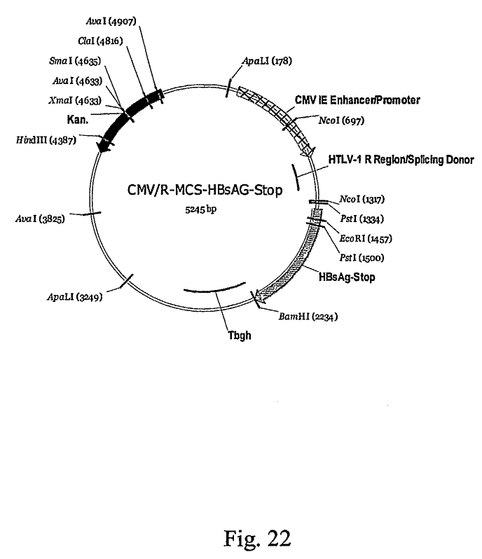

K562 cells labeled with either HBsAG-specific NF5 or MPR-specific 2F5 and 4E10 or negative control HIVIgG were used instead of B-cells from HIV positive subjects. NF5-labeled cells specifically bound to HBsAG particles. HIV IgG-labeled cells failed to bind either the nile red HBsAG or HBsAG-MPR particles. 2F5 and 4E10 labeled cells specifically bound to nile red HBsAG-MPR particles. This specific binding of cells labeled with unique antibody indicates that this technique can be used to identify HIV-1 specific B-cells. See FIG. 16.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

TABLE 1

| | |
|---|---|
| Primer Name | Primer Sequence |

| | |
|---|---|
| SAg-Forward | 5′ GGAGCTCGTCGA GAGCAA 3′ |
| | (SEQ ID NO: 38) |
| SAg-Reverse | 5′ GC TCT AGA CCC GA T GTA CAC CCA 3′ |
| | (SEQ ID NO: 59) |
| MPR Forward | 5′ GC TCT AGA AAC GAG CAG GAG CTG CTG |
| | 3′ |
| | (SEQ ID NO: 41) |
| MPR Reverse | 5′ CGC GGA TCC TCA CCC CTT GAT GTA |
| | CCA CAG CCA CTT 3′ |
| | (SEQ ID NO: 42) |
| MPR-Foldon | 5′ CGC GGA TCC TCA ATG GTG ATG GTG |
| Rev | ATG GTG GGG 3′ |
| | (SEQ ID NO: 43) |
| C-heptad-MPR | 5′ GC TCT AGA GCC GTG GAG CGG TAC CTG |
| Forward | 3′ |
| | (SEQ ID NO: 44) |
| MPR-Tm5 | 5′ CTCGGATCCTCAAATCATGATGAAAATCTTGAT |
| Reverse | 3′ |
| | (SEQ ID NO: 45) |
| MPR-Tm10 | 5′ CTCGGATCCTCACACCAGGCCACCAACAAT 3′ |
| Reverse | (SEQ ID NO: 46) |
| MPR-Tm15 | 5′ CTCGGATCCTCACACCAGCCTCAGGCCCAC 3′ |
| Reverse | (SEQ ID NO: 47) |
| MPR-Tm23-C9 | 5′ CTCGGATCCTCAGGCGGGCGC 3′ |
| Reverse | (SEQ ID NO: 48) |
| AgeI | 5′ CCCTGCAAGACCTGCACC |
| Forward | ACCACCGGTCAGGGCAACTCCAAGTTCCCC 3′ |
| | (SEQ ID NO: 49) |
| AgeI | 5′ GGGGAACTTG GAGTTGCCCT GACCGGTGGT |
| reverse | GGTGCAGGTC TTGCAGGG 3′ |
| | (SEQ ID NO: 50) |
| MPR AgeI | 5′ GGC ACC GGT AAC GAG CAG GAG CTG |
| Forward | CTG 3′ |
| | (SEQ ID NO: 51) |
| MPR AgeI | 5′ GGC ACC GGT CCC CTT GAT GTA CCA |
| Reverse | CAG CCA CTT 3′ |
| | (SEQ ID NO: 52) |
| MPRSAG | 5′ AGC GAA TTC AAC GAG CAG GAG CTG |
| Forward | CTG 3′ |
| | (SEQ ID NO: 53) |
| MPR SAG | 5′ CGC GGA TCC TCA CCC GA T GTA CAC |
| Reverse | CCA 3′ |
| | (SEQ ID NO: 54) |
| SAGMPR RI | 5′ CAG GAA GCC GGA GGT GATGAA CCC CTT |
| forward | GAT GTA CCA CAG CCA CTT 3′ |
| | (SEQ ID NO: 55) |
| SAG MPR RI | 5′ AAG TGG CTG TGG TAC ATC AAG GGG |
| Reverse | TTC ATC ACC TCC GGC TTC CTG 3′ |
| | (SEQ D NO: 56) |

TABLE 2

| Human Sera | HBsAg-MPR | HBsAg | MPR reactivity | Neutralization |
|---|---|---|---|---|
| 1 | +/− | +/− | Negative | Broad |
| 4 | +/− | +/− | Negative | Weak |
| 5 | +/− | +/− | Negative | Weak |
| 6 | +++ | ++ | Not clear | Moderate |
| 7 | ++ | ++ | Negative | Moderate |
| 20 | ++++ | ++ | Positive | Broad |
| 30 | ++++ | ++ | Positive | Broad |
| 45 | ++++ | ++ | Positive | Broad |
| HIVIg | − | − | Negative | Broad |

It will be apparent that the precise details of the methods or compositions described may be varied or modified without departing from the spirit of the described invention. We claim all such modifications and variations that fall within the scope and spirit of the claims below.

1. A monomeric fusion protein comprising the following elements linked in an N-terminal to C-terminal direction:
(a) a hepatitis B surface antigen;
(b) a linear linking peptide; and,
(c) an antigenic polypeptide comprising the amino acid sequence of SEQ ID NO: 1 (NEX₁X₂LLX₃LDKWASLWNWFDITNWLWYIK), wherein the antigenic peptide is between 28 and 150 amino acids in length,
wherein X₁, X₂ and X₃ are any amino acid, and wherein a plurality of the monomeric fusion proteins form a self-aggregating multimeric ring structure upon expression in a host cell.

2. The monomeric fusion protein of claim 1, wherein the antigenic peptide comprises the amino acid set forth as one of:

| | | |
|---|---|---|
| a) | SEQ ID NO: 2 | (NEQELLALDKWASLWNWFDITNWLWYIK); |
| b) | SEQ ID NO: 3 | (NEQDLLALDKWASLWNWFDITNWLWYIK); |
| c) | SEQ ID NO: 4 | (NEQDLLALDKWANLWNWFDISNWLWYIK); |
| d) | SEQ ID NO: 5 | (NEQDLLALDKWANLWNWFNITNWLWYIR); |
| e) | SEQ ID NO: 6 | (NEQELLELDKWASLWNWFDITNWLWYIK); |
| f) | SEQ ID NO: 7 | (NEKDLLALDSWKNLWNWFDITNWLWYIK); |
| g) | SEQ ID NO: 8 | (NEQDLLALDSWENLWNWFDITNWLWYIK); |
| h) | SEQ ID NO: 9 | (NEQELLELDKWASLWNWFSITQWLWYIK); |
| i) | SEQ ID NO: 10 | (NEQELLALDKWASLWNWFDISNWLWYIK); |
| j) | SEQ ID NO: 11 | (NEQDLLALDKWDNLWSWFTITNWLWYIK); |
| k) | SEQ ID NO: 12 | (NEQDLLALDKWASLWNWFDITKWLWYIK); |
| l) | SEQ ID NO: 13 | (NEQDLLALDKWASLWNWFSITNWLWYIK); |
| m) | SEQ ID NO: 14 | (NEKDLLELDKWASLWNWFDITNWLWYIK); |
| n) | SEQ ID NO: 15 | (NEQEILALDKWASLWNWFDISKWLWYIK); |
| o) | SEQ ID NO: 16 | (NEQDLLALDKWANLWNWFNISNWLWYIK); |
| p) | SEQ ID NO: 17 | (NEQDLLALDKWASLWSWFDISNWLWYIK); |
| q) | SEQ ID NO: 18 | (NEKDLLALDSWKNLWSWFDITNWLWYIK); |
| r) | SEQ ID NO: 19 | (NEQELLQLDKWASLWNWFSITNWLWYIK); |
| s) | SEQ ID NO: 20 | (NEQDLLALDKWASLWNWFDISNWLWYIK); |
| t) | SEQ ID NO: 21 | (NEQELLALDKWASLWNWFDISNWLWYIR); |
| or | | |
| u) | SEQ ID NO: 22 | (NEQELLELDKWASLWNWFNITNWLWYIK). |

3. A monomeric fusion protein comprising the following elements linked in an N-terminal to C-terminal direction:
(a) a hepatitis B surface antigen;
(b) a linear linking peptide; and,
(c) an antigenic polypeptide comprising one to five repeats of the amino acid sequence of SEQ ID NO:23 (consensus of 2F5 epitope) (EQXLLXLDKWASLWGG), wherein the antigenic polypeptide does not include amino acids 1 to 500 of a gp160 amino acid sequence (SEQ ID NO: 25), and wherein X is any amino acid.

4. The monomeric fusion protein of claim 3, wherein X is glutamine (E). comprises SEQ ID NO: 24 (EQELLELDKWASLWGG) SEQ ID NO:24.

5. The monomeric fusion protein of claim 1, further comprising at the C-terminus at least five consecutive hydrophobic amino acid residues.

6. The monomeric fusion protein of claim 5, wherein the hydrophobic residues comprise the amino acid sequence of SEQ ID NO:26 (IFIMI).

7. The monomeric fusion protein of claim 5, wherein the hydrophobic residues comprise the amino acid sequence of SEQ ID NO:27 (IFIMIVGGLV).

8. The monomeric fusion protein of claim 5, wherein the hydrophobic residues comprise the amino acid sequence of SEQ ID NO:28 (IFIMIVGGLVGLRLV).

9. The monomeric fusion protein of claim 5, wherein the hydrophobic residues comprise the amino acid sequence of SEQ ID NO:29 (IFIMIVGGLVGLRLVFSIETGG).

10. The monomeric fusion protein of claim 1, further comprising at the C-terminus at least five consecutive basic amino acid residues.

11. The monomeric fusion protein of claim 1, wherein the hepatitis B surface antigen is encoded by the nucleic acid sequence of SEQ ID NO:30.

12. The monomeric fusion protein of claim 1, further comprising an HIV-specific T-helper cell epitope.

13. The monomeric fusion protein of claim 12, wherein the HIV-specific T-helper cell epitope is the amino acid sequence of SEQ ID NO:32 or 33.

14. An isolated nucleic acid molecule encoding the monomeric fusion protein of claim 1.

15. An isolated nucleic acid molecule encoding the monomeric fusion protein of claim 12.

16. The isolated nucleic acid molecule of claim 14 operably linked to a promoter.

17. The isolated nucleic acid molecule of claim 14, further comprising a nucleotide sequence encoding at least one CAAX (SEQ ID NO:34) sequence.

18. A host cell transformed with the nucleic acid molecule of claim 14.

19. A viral-like particle produced by the host cell of claim 18.

20. The viral-like particle of claim 19, further comprising at least one TLR ligand.

21. A composition comprising the viral-like particles of claim 19 in a pharmaceutically acceptable carrier.

22. A composition comprising the monomeric fusion protein of claim 1, a polymeric form thereof, or a nucleic acid encoding the monomeric fusion protein in a pharmaceutically acceptable carrier.

23. The composition of claim 22, comprising a therapeutically effective amount of the monomeric fusion protein of claim 1, a polymeric form thereof, or a nucleic acid encoding the monomeric fusion protein and an adjuvant.

24. A method for inhibiting HIV infection in a subject, comprising administering a therapeutically effective amount of the composition of claim 21 to the subject, thereby inhibiting HIV infection.

25. A method for inducing an immune response to HIV in a subject, comprising administering the composition of claim 21 to the subject, thereby inducing the immune response.

26. The method of claim 23, wherein the immune response comprises the induction of neutralizing antibodies to HIV.

27. A method for inhibiting HIV infection in a subject, comprising:
administering a therapeutically effective amount of the monomeric fusion protein of claim 1, or a polymeric form thereof, to the subject,
thereby inhibiting HIV infection.

28. The method of claim 27, further comprising administering an adjuvant to the subject.

29. A method for diagnosing HIV infection in a subject, comprising:
contacting a sample from the subject with a monomeric fusion protein of claim 1 or a polymeric form thereof; and
detecting whether antibody present in the sample binds to the protein,
wherein binding of an antibody to the monomeric fusion protein of the polymeric form thereof indicates that the subject has an HIV infection.

30. The method of claim 29, wherein the sample is a serum sample.

31. A method for identifying a B cell that produces antibodies that bind to gp41, comprising:
contacting supernatant from the B cell with the monomeric fusion protein of claim 1 and
determining if the B cell secretes an antibody binds to gp41.


SEQUENCE LISTING (70 sequences)

SEQ ID NO: 1 - protein, 28 aa, Artificial sequence
  Consensus sequence for MPR region
NEXXLLXLDKWASLWNWFDITNWLWYIK

SEQ ID NO: 2 - protein, 28 aa, Artificial sequence
  MPR consensus sequence from HIV-1
NEQELLALDKWASLWNWFDITNWLWYIK

SEQ ID NO: 3 - protein, 28 aa, Artificial sequence
  MPR ancestral sequence for HIV-1 clade M
NEQDLLALDKWASLWNWFDITNWLWYIK

SEQ ID NO: 4 - protein, 28 aa, Artificial sequence
  MPR consensus sequence from HIV-1 clade A1
NEQDLLALDKWANLWNWFDISNWLWYIK

SEQ ID NO: 5 - protein, 28 aa, Artificial sequence
  MPR consensus sequence from HIV-1 clade A2
NEQDLLALDKWANLWNWFNITNWLWYIR

SEQ ID NO: 6 - protein, 28 aa, Artificial sequence
  MPR consensus sequence from HIV-1 clade B
NEQELLELDKWASLWNWFDITNWLWYIK

SEQ ID NO: 7 - protein, 28 aa, Artificial sequence
  MPR concensus sequence for HIV-1 clade C
NEKDLLALDSWKNLWNWFDITNWLWYIK

SEQ ID NO: 8 - protein, 28 aa, Artificial sequence
  MPR ancestral sequence for HIV-1 clade C
NEQDLLALDSWENLWNWFDITNWLWYIK

SEQ ID NO: 9 - protein, 28 aa, Artificial sequence
  MPR consensus sequence for HIV-1 clade D
NEQELLELDKWASLWNWFSITQWLWYIK

SEQ ID NO: 10 - protein, 28 aa, Artificial sequence
  MPR consensus sequence for HIV-1 clade F1
NEQELLALDKWASLWNWFDISNWLWYIK

SEQ ID NO: 11 - protein, 28 aa, Artificial sequence
  MPR consensus sequence for HIV-1 clade F2
NEQDLLALDKWDNLWSWFTITNWLWYIK

SEQ ID NO: 12 - protein, 28 aa, Artificial sequence
  MPR consensus sequence for HIV-1 clade G
NEQDLLALDKWASLWNWFDITKWLWYIK

SEQ ID NO: 13 - protein, 28 aa, Artificial sequence
  MPR consensus sequence for HIV-1 clade H
NEQDLLALDKWASLWNWFSITNWLWYIK

SEQ ID NO: 14 - protein, 28 aa, Artificial sequence
  MPR consensus sequence for HIV-1 clade AE
NEKDLLELDKWASLWNWFDITNWLWYIK

SEQ ID NO: 15 - protein, 28 aa, Artificial sequence
  MPR consensus sequence for HIV-1 clade AB
NEQEILALDKWASLWNWFDISKWLWYIK

SEQ ID NO: 16 - protein, 28 aa, Artificial sequence
  MPR consensus sequence for HIV-1 clade 04CPX
NEQDLLALDKWANLWNWFNISNWLWYIK

SEQ ID NO: 17 - protein, 28 aa, Artificial sequence
  MPR consensus sequence for HIV-1 clade 06CPX
NEQDLLALDKWASLWSWFDISNWLWYIK

SEQ ID NO: 18 - protein, 28 aa, Artificial sequence
  MPR consensus sequence for HIV-1 clade 08BC
NEKDLLALDSWKNLWSWFDITNWLWYIK

SEQ ID NO: 19 - protein, 28 aa, Artificial sequence
  MPR consensus sequence for HIV-1 clade 10CD
NEQELLQLDKWASLWNWFSITNWLWYIK

SEQ ID NO: 20 - protein, 28 aa, Artificial sequence
  MPR consensus sequence for HIV-1 clade 11CPX
NEQDLLALDKWASLWNWFDISNWLWYIK

SEQ ID NO: 21 - protein, 28 aa, Artificial sequence
  MPR consensus sequence for HIV-1 clade 12BF
NEQELLALDKWASLWNWFDISNWLWYIR

SEQ ID NO: 22 - protein, 28 aa, Artificial sequence
  MPR consensus sequence for HIV-1 clade 14BG
NEQELLELDKWASLWNWFNITNWLWYIK

SEQ ID NO: 23 - protein, 16 aa, Artificial sequence
  Consensus sequence for 2F5 epitope
EQXLLXLDKWASLWGG

SEQ ID NO: 24 - protein, 16 aa, Artificial sequence
  2F5 epitope
EQELLELDKWASLWGG

SEQ ID NO: 25 - protein, 857 aa, Human immunodeficiency virus type 1
MRAREMRRNWQDLWKWGIMLLGMWMICSATENLWVTVYYGVPVWKEATTTLFCASDAKAYDKEVHNVWATHASVPTDPNPQEVVLANVTENFNMWKNNMVDQMHEDIISLWDESLKPCVKLTPLCVTLMCANVNVTNKANMTGPNNTSWEKMEGEIKNCSFNITTNIKDKREKKYALFYALDLVSMKDNADISKTNNSYRLIHCNTSTITQACPKVSFEPIPIHYCAPAGFAILKCNNKKFNGTGPCTNVSTVQCTHGIKPVVSTQLLLNGSLAEEEIVIRSENLTDNAKNIIVQLNKSIEINCTRPNNNTRQSISIGPGRALYTTGQIIGDIRQAYCNLSKVSWNNTLKQIAAKLREHFNKTIIFKSSSGGDPEIVTHSFNCGREFFYCNTSKLFNSTWGLNQTANHEGNDTTITLPCRIKQIINMWQKVGKAMYAPPIAGRIACSSHITGLLLTRDGGNDTNNETFRPGGGNMRDNWRSELYKYKVVKIKPLGIAPTKAKRRVVQREKRAVGTLGAMFLGFLGAAGSTMGAASVTLTVQARQLLSGIVQQQNNLLRAIEAQQHLLQLTVWGIKQLQARILAVERYLQDQQLLGIWGCSGKLICTTAVPWNVSWSNKSHTEIWDNMTWMEWEKEIDNYTSIIYTLLETSQNQQEKNEQELLELDKWASLWNWFSISNWLWYIKIFIMIAGGLIGLRIVFAVLSIVNRVRQGYSPLSFQTHLPAPRGPDRPEGIEEEGGERGRDRSSHLARGFSIPIWDDLWTLCLFSYHILRDLLLTVARIVEILRRRGWEVLKYWWNLLQYWSQELKNSAVSLLNTTAIVVGEGTDRIIEVVQRFLRAVLHIPRRIRQGLERALL

SEQ ID NO: 26 - protein, 5 aa, Artificial sequence
  Hydrophobic amino acid group
IFIMI

SEQ ID NO: 27 - protein, 10 aa, Artificial sequence
  Hydrophobic amino acid group
IFIMIVGGLV

SEQ ID NO: 28 - protein, 15 aa, Artificial sequence
  Hydrophobic amino acid group
IFIMIVGGLVGLRLV

SEQ ID NO: 29 - protein, 22 aa, Artificial sequence
  Hydrophobic amino acid group
IFIMIVGGLVGLRLVFSIETGG

SEQ ID NO: 30 - DNA, 684 bp, Hepatitis B virus
gaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctaga

SEQ ID NO: 31 - protein, 228 aa, Hepatitis B virus
EFITSGFLGPLLVLQAGFFLLTRILTIPQSLDSWWTSLNFLGGSPVCLGQNSQSPTSNHSPTSCPPICPGYRWMCLRRFIIFLFILLLCLIFLLVLLDYQGMLPVCPLIPGSTTTSTGPCKTCTTPAQGNSKFPSCCCTKPTDGNCTCIPIPSSWAFAKYLWEWASVRFSWLSLLVPFVQWFVGLSPTVWLSAIWMMWYWGPSLYSIVSPFIPLLPIFFCLWVYIGSR

SEQ ID NO: 32 - protein, 15 aa, Artificial sequence
  T helper cell epitope
ASLWNWFNITNWLWY

SEQ ID NO: 33 - protein, 15 aa, Artificial sequence
  T helper cell epitope
IKLFIMIVGGLVGLR

SEQ ID NO: 34 - protein, 4 aa, Artificial sequence
  Synthetic peptide
CXXX

SEQ ID NO: 35 - protein, 7 aa, Artificial sequence
  Core of 2F5 epitope
ELDKWAS

SEQ ID NO: 36 - protein, 6 aa, Artificial sequence
  Core of 4E10 epitope
NWFDIT

SEQ ID NO: 37 - protein, 4 aa, Artificial sequence
  Synthetic linker sequence
GPGP

SEQ ID NO: 38 - DNA, 18 bp, Artificial sequence
  Primer
ggagctcgtcgacagcaa

SEQ ID NO: 39 - DNA, 23 bp, Artificial sequence
  Primer
gctctagacccgatgtagaccca

SEQ ID NO: 40 - protein, 16 aa, Artificial sequence
  Peptide used in ELISA
NEQELLELDKWASLWN

SEQ ID NO: 41 - DNA, 26 bp, Artificial sequence
  Primer
gctctagaaacgagcaggagctgctg

SEQ ID NO: 42 - DNA, 36 bp, Artificial sequence
  Primer
cgcggatcctcaccccttgatgtaccacagccactt

SEQ ID NO: 43 - DNA, 33 bp, Artificial sequence
  Primer
cgcggatcctcaatggtgatggtgatggtgggg

SEQ ID NO: 44 - DNA, 26 bp, Artificial sequence
  Primer
gctctagagccgtggagcggtacctg

SEQ ID NO: 45 - DNA, 33 bp, Artificial sequence
  Primer
ctcggatcctcaaatcatgatgaaaatcttgat

SEQ ID NO: 46 - DNA, 30 bp, Artificial sequence
  Primer
ctcggatcctcacaccaggccaccaacaat

SEQ ID NO: 47 - DNA, 30 bp, Artificial sequence
  Primer
ctcggatcctcacaccagcctcaggcccac

SEQ ID NO: 48 - DNA, 21 bp, Artificial sequence
  Primer
ctcggatcctcaggcgggcgc

SEQ ID NO: 49 - DNA, 48 bp, Artificial sequence
  Primer
ccctgcaagacctgcaccaccaccggtcagggcaactccaagttcccc

SEQ ID NO: 50 - DNA, 48 bp, Artificial sequence
  Primer
ggggaacttggagttgccctgaccggtggtggtgcaggtcttgcaggg

SEQ ID NO: 51 - DNA, 27 bp, Artificial sequence
  Primer
ggcaccggtaacgagcaggagctgctg

SEQ ID NO: 52 - DNA, 33 bp, Artificial sequence
  Primer
ggcaccggtccccttgatgtaccacagccactt

SEQ ID NO: 53 - DNA, 27 bp, Artificial sequence
  Primer
agcgaattcaacgagcaggagctgctg

SEQ ID NO: 54 - DNA, 45 bp, Artificial sequence
  Primer
caggaagccggaggtgatgaaccccttgatgtaccacagccactt

SEQ ID NO: 55 - DNA, 45 bp, Artificial sequence; identical to SEQ ID NO: 54
  Primer

SEQ ID NO: 56 - DNA, 45 bp, Artificial sequence
  Primer
aagtggctgtggtacatcaaggggttcatcacctccggcttcctg

SEQ ID NO: 57 - protein, 5 aa, Artificial sequence
  Group of basic amino acid residues
HRKKR

SEQ ID NO: 58 - protein, 10 aa, Artificial sequence
  Group of basic amino acid residues
HRKRHKRRKH

SEQ ID NO: 59 - DNA, 23 bp, Artificial sequence
  Primer
gctctagacccgatgtacaccca

SEQ ID NO: 60 - DNA, 5573 bp, Artificial sequence
  Construct CMV/R-MCS-HBsAg-C-heptad-MPR-FL
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagagccgtggagcggtacctgcgagaccagcagctgctgggcatctggggctgcagcggcaagctgatctgcaccaccaccgtgccctggaacaccagctggagcaacaagagcctgaacgagatctgggacaacatgacctggatgaagtgggagcgggagatcgacaactacacccacatcatctacagcctgatcgagcagagccagaaccagcaggagaagaacgagcaggagctgctggccctggacaagtgggccagcctgtggaactggtttgacatcaccaagtggctgtggtacatcaagggggggggttacatcccggaagctcctcgagacggtcaggcttacgttcgtaaagacggtgaatgggttctgctgtctaccttcctgcccccccaccatcaccatcaccattgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 61 - DNA, 5338 bp, Artificial sequence
  Construct CMV/R-MCS-HBsAg125-MPR-128
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccaccaccggtaacgagcaggagctgctggccctggacaagtgggcctccctgtggaactggttcgacatcaccaagtggctgtggtacatcaaggggaccggtcagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtgatctagaaacgagcaggagctgctggccctggacaagtgggcctccctgtggaactggttcgacatcaccaagtggctgtggtacatcaaggggtgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 62 - DNA, 5242 bp, Artificial sequence
  Construct CMV/R-MCS-HBsAg-MPR
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagaaacgagcaggagctgctggccctggacaagtgggccagcctgtggaactggtttgacatcaccaagtggctgtggtacatcaaggggtgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 63 - DNA, 5269 bp, Artificial sequence
  Construct CMV/R-MCS-HBsAg-MPR10
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagaaacgagcaggagctgctggccctggacaagtgggccagcctgtggaactggtttgacatcaccaagtggctgtggtacatcaagattttcatcatgattgttggtggcctggtgtgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 64 - DNA, 5332 bp, Artificial sequence
  Construct CMV/R-MCS-HBsAg-MPR-Tm-C9
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagaaacgagcaggagctgctggccctggacaagtgggccagcctgtggaactggtttgacatcaccaagtggctgtggtacatcaagattttcatcatgattgttggtggcctggtgggcctgaggctggtgttcagcattgagacgggcggcaccgagacctcccaggtggcgcccgcctgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 65 - DNA, 5233 bp, Artificial sequence
  Construct CMV/R-MCS-MPR-HBsAg
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttatgtctggttctcgctcaaaaacttcccggaaatgacaacaacagcgaattcaacgagcaggagctgctggccctggacaagtgggcctccctgtggaactggttcgacatcaccaagtggctgtggtacatcaagatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 66 - DNA, 5366 bp, Artificial sequence
  Construct CMV/R-HBsAg-MPR-FL
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcggtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagaaaccagcaggagaagaacgagcaggagctgctggccctggacaagtgggccagcctgtggaactggtttgacatcaccaagtggctgtggtacatcaagggggggggttacatcccggaagctcctcgagacggtcaggcttacgttcgtaaagacggtgaatgggttctgctgtctaccttcctgcccccccaccatcaccatcaccattgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 67 - DNA, 5464 bp, Artificial sequence
  Construct CMV/R-MCS-HBsAg-C-heptad-MPR
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagagccgtggagcggtacctgcgagaccagcagctgctgggcatctggggctgcagcggcaagctgatctgcaccaccaccgtgccctggaacaccagctggagcaacaagagcctgaacgagatctgggacaacatgacctggatgaagtgggagcgggagatcgacaactacacccacatcatctacagcctgatcgagcagagccagaaccagcaggagaagaacgagcaggagctgctggccctggacaagtgggccagcctgtggaactggtttgacatcaccaagtggctgtggtacatcaaggggtgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 68 - DNA, 5254 bp, Artificial sequence
  Construct CMV/R-MCS-HBsAg-MPR5
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagaaacgagcaggagctgctggccctggacaagtgggccagcctgtggaactggtttgacatcaccaagtggctgtggtacatcaagattttcatcatgatttgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 69 - DNA, 5284 bp, Artificial sequence
  Construct CMV/R-MCS-HBsAg-MPR15
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtctagaaacgagcaggagctgctggccctggacaagtgggccagcctgtggaactggtttgacatcaccaagtggctgtggtacatcaagattttcatcatgattgttggtggcctggtgggcctgaggctggtgtgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 70 - DNA, 5245 bp, Artificial sequence
  Construct CMV/R-MCS-HBsAg-STOP
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccatcggctcgcatctctccttcacgcgcccgccgccttacctgaggccgccatccacgccggttgagtcgcgttctgccgcctcccgcctgtggtgcctcctgaactacgtccgccgtctaggtaagtttagagctcaggtcgagaccgggcctttgtccggcgctcccttggagcctacctagactcagccggctctccacgctttgcctgaccctgcttgctcaactctagttaacggtggagggcagtgtagtctgagcagtactcgttgctgccgcgcgcgccaccagacataatagctgacagactaacagactgttcctttccatgggtcttttctgcagtcaccgtcgtcgacagcaaaagcaggggataattctattaaccatgaagactatcattgctttgagctacattttctgtctggttttcgcccaagaccttccaggaaatgacaacaacagcgaattcatcacctccggcttcctgggccccctgctggtcctgcaggccgggttcttcctgctgacccgcatcctcaccatcccccagtccctggactcgtggtggacctccctcaactttctggggggctcccccgtgtgtctgggccagaactcccagtcccccacctccaaccactcccccacctcctgcccccccatctgccccggctaccgctggatgtgcctgcgccgcttcatcatcttcctgttcatcctgctgctgtgcctgatcttcctgctggtgctgctggactaccagggcatgctgcccgtgtgccccctgatccccggctccaccaccacctccaccggcccctgcaagacctgcaccacccccgcccagggcaactccaagttcccctcctgctgctgcaccaagcccaccgacggcaactgcacctgcatccccatcccctcctcctgggccttcgccaagtacctgtgggagtgggcctccgtgcgcttctcctggctgtccctgctggtgcccttcgtgcagtggttcgtgggcctgtcccccaccgtgtggctgtccgccatctggatgatgtggtactggggcccctccctgtactccatcgtgtcccccttcatccccctgctgcccatcttcttctgcctgtgggtgtacatcgggtgatctagaaacgagcaggagctgctggccctggacaagtgggcctccctgtggaactggttcgacatcaccaagtggctgtggtacatcaaggggtgaggatccagatctgctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgggtacccaggtgctgaagaattgacccggttcctcctgggccagaaagaagcaggcacatccccttctctgtgacacaccctgtccacgcccctggttcttagttccagccccactcataggacactcatagctcaggagggctccgccttcaatcccacccgctaaagtacttggagcggtctctccctccctcatcagcccaccaaaccaaacctagcctccaagagtgggaagaaattaaagcaagataggctattaagtgcagagggagagaaaatgcctccaacatgtgaggaagtaatgagagaaatcatagaattttaaggccatcatggccttaatcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactcggggggggggggcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc